(19) United States
(12) Patent Application Publication
(10) Pub. No.: US 2026/0345671 A1
(43) Pub. Date: Nov. 5, 2026

(54) Methods of Treating Conditions Responsive to Nitric Oxide Therapy

(71) Applicant: SaNOtize Research and Development Corp., Vancouver (CA)

(72) Inventors: Gilly Regev, Vancouver (CA); Christopher C. Miller, North Vancouver (CA)

(21) Appl. No.: 19/575,640

(22) Filed: Mar. 23, 2026

Related U.S. Application Data
(63) Continuation of application No. 18/016,655, filed on Jul. 16, 2021, now abandoned.
(60) Provisional application No. 63/052,805, filed on Jul. 16, 2020.

(51) Int. Cl. A61K 33/00; A61K 9/08; A61K 47/12; A61P 17/02; A61P 31/10

(52) U.S. Cl. CPC A61K 33/00; A61K 9/08; A61K 47/12; A61P 17/02; A61P 31/10

(57) ABSTRACT

Compositions and methods of treating conditions in a subject that are responsive to nitric oxide (NO) treatment are disclosed and described. Such compositions can include a combination of a nitric oxide releasing compound, an acidifying agent, and a carrier in amounts that release from about 1 ppm*min/mL to about 500 ppm*min/mL NO based on 1 mL of the composition when measured by releasing NO from the composition for a period of 30 minutes into a flow of substantially inert carrier gas at a flow rate of 1 liter per minute and a pressure of 1 atmosphere, coupled to a chemiluminescence detector.

### PRIORITY DATA

This application is a continuation of U.S. patent application Ser. No. 18/016,655, filed Jan. 17, 2023, which is a 371 U.S. National Stage entry of PCT Application Serial No. PCT/IB2021/000505, filed Jul. 16, 2021, which claims the benefit of U.S. Provisional Application Ser. No. 63/052,805 filed Jul. 16, 2020, each of which is incorporated herein by reference.

### BACKGROUND

Nitric oxide (NO) is a diatomic molecule including a single nitrogen atom and a single oxygen atom. The NO molecule is quite unstable and has a free radical-like nature. NO is easily oxidized into nitrogen dioxide (NO₂). Accordingly, NO can be difficult to produce and store or use in controlled quantities. Within the human body, nitric oxide can be produced by nitric oxide synthase enzymes (NOS). Many physiological processes and pathological process in the body can involve NO. For example, a low level of NO in the blood can encourage vasodilation to prevent ischemic damage, can help with wound healing, and can be an effective antimicrobial agent. Conversely, a high level of NO in the blood can lead to tissue toxicity and can contribute to inflammatory conditions such as septic shock, diabetes, and arthritis.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure; and, wherein:

FIG. 1 a chart depicting differences in exposure between naturally occurring NO respiration, INOmax, nitric oxide nitric oxide nasal spray (NONS) prevention, and NONS treatment in accordance with an example;

FIG. 2a illustrates application of bacteria mixture to a porcine subject in accordance with an example;

FIG. 2b depicts the concentration of various bacteria types for a control, a nitric oxide releasing gel (NORG) treatment, and a nitric oxide releasing solution (NORS)+NORG treatment in accordance with an example;

FIG. 3a illustrates a wart on a foot prior to commencement of treatment in accordance with an example;

FIG. 3b illustrates resolution of a wart on a foot after treatment in accordance with an example;

FIG. 3c illustrates warts on a foot on day 1 after commencement of treatment in accordance with an example;

FIG. 3d illustrates warts on a foot on day 7 after commencement of treatment in accordance with an example;

FIG. 3e illustrates warts on a foot on day 14 after commencement of treatment in accordance with an example;

FIG. 3f illustrates warts on a foot on day 28 after commencement of treatment in accordance with an example;

FIG. 4a illustrates nail fungus before commencement of treatment in accordance with an example;

FIG. 4b illustrates resolution of nail fungus after treatment in accordance with an example;

FIG. 4c illustrates toenail fungus before commencement of treatment in accordance with an example;

FIG. 4d illustrates toenail fungus after 1 day of treatment in accordance with an example;

FIG. 4e illustrates toenail fungus after 3 weeks after the commencement of treatment in accordance with an example;

FIG. 4f illustrates toenail fungus 2 months after the commencement of treatment in accordance with an example;

FIG. 4g illustrates toenail fungus 42 months after the commencement of treatment in accordance with an example;

FIG. 4h illustrates toenail fungus 9 months after the commencement of treatment in accordance with an example;

FIG. 4i illustrates toenail fungus after 1 day of treatment in accordance with an example;

FIG. 4j illustrates toenail fungus 28 days after the commencement of treatment in accordance with an example;

FIG. 4k illustrates toenail fungus 63 days after the commencement of treatment in accordance with an example;

FIG. 4l illustrates toenail fungus 77 days after the commencement of treatment in accordance with an example; and

FIG. 4m illustrates the resolution of toenail fungus 120 days after the commencement of treatment in accordance with an example.

Reference will now be made to the exemplary embodiments illustrated, and specific language will be used herein to describe the same. It will nevertheless be understood that no limitation of the scope of the invention is thereby intended.

### DESCRIPTION OF EMBODIMENTS

Although the following detailed description contains many specifics for the purpose of illustration, a person of ordinary skill in the art will appreciate that many variations and alterations to the following details can be made and are considered to be included herein. As such, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used have the same meaning as commonly understood by one of ordinary skill in the art to which this disclosure belongs.

In this written description, the singular forms “a,” “an” and “the” provide express support for plural referents unless the context clearly dictates otherwise. Thus, for example, reference to “a particle” includes a plurality of particles.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described as comprising a series of steps, the order of such steps as presented is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein, “subject” refers to a mammal that may benefit from the administration of NORS or NO. In one aspect the mammal may be a human.

As used herein, the terms “treat,” “treatment,” or “treating” when used in conjunction with the administration of NORS or NO, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. In one example, a composition can be a solution that releases NO.

A “kit” can mean a package or container that includes a composition or dosage form along with instructions regarding application or administration of the composition or dosage form according to a given regimen or within specified time and amount parameters to treat one or more specific indications. For example, a kit could include a NORS composition, or the components thereof, in a specific volume or amount along with a set of instructions on appropriate administration of the NORS to a subject in order to treat a given condition (e.g. indication). Instructions may include direction for a single type of administration or indication, or for multiple types of administration or indications. Additionally, the amount and form of the NORS composition in the kit can be suitable for a single administration for treatment of a single indication, multiple administrations creating a regimen for one indication, or single or multiple administrations for multiple indications. For example, a composition or dosage form of a NORS composition can be provided in the kit along with instructions for applying the dosage form in terms of amount and volume that is suitable to treat a plurality of indications, such as skin conditions such as acne, or wound healing, or respiratory therapy, or further yet for improving immunity in the sinus and throat areas.

As used herein “NORS” refers to a nitric oxide (NO) releasing solution, composition or substance, which includes an acidified nitrite capable of releasing NO. In one aspect, NO released from NORS may be a gas. A NORS can be in a variety of physical forms, such as a liquid, cream, gel, or others.

As used herein, “nitric oxide releasing compound” and “nitric oxide releasing agent” can be used interchangeably and it is to be understood that recitation of one phrase in this written description provides express support for the other. Such terms refer to a compound, agent, or molecule that can liberate nitric oxide upon reaction with another compound or agent, such as an acid. Nitric oxide (NO) can be liberated as a gas (e.g. gNO). Furthermore, the use of the term “nitrite” in this written description can be used to refer generally to either a “nitric oxide releasing compound” or “nitric oxide releasing agent”, or can specifically refer to the compound of NO₂ as is traditionally known in the chemical arts and express support for use of the terms interchangeably is hereby included.

As used herein a “therapeutic agent” refers to an agent that can have a beneficial or positive effect on a subject when administered to the subject in an appropriate or effective amount. In one aspect, NO can be a therapeutic agent. In another aspect, therapeutic agents can include non-NORS agents with physiologic activity, such as antibiotics, antihistamines, antivirals, antimicrobials, biological molecules, such as siRNA, cDNA, steroids, vasodilators, vasoconstrictors, analgesics, anti-inflammatories, etc. In some aspects, therapeutic agent can be used interchangeably with “active agent” or “drug”.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a “dosing regimen” or “regimen” such as “treatment dosing regimen,” or a “prophylactic dosing regimen” refers to how, when, how much, and for how long a dose of a composition can or should be administered to a subject in order to achieve an intended treatment or effect.

As used herein, the terms “release” and “release rate” are used interchangeably to refer to the discharge or liberation, or rate thereof, of a substance, including without limitation a therapeutic agent, such as NO, from the dosage form or composition containing the substance. In one example, a therapeutic agent may be released in vitro. In another aspect, a therapeutic agent may be released in vivo.

As used herein, “immediate release” or “instant release” can be used interchangeably and refer to immediate or near immediate (i.e. uninhibited or unrestricted) release of an agent or substance, including a therapeutic agent, such as NO, from a composition or formulation.

As used herein, the term “controlled release” refers to non-immediate release of an agent or substance, including a therapeutic agent, such as NO, from a composition or formulation. Examples of specific types of controlled release include without limitation, extended or sustained release and delayed release. Any number of control mechanisms or components can be used to create a controlled release effect, including formulation ingredients or constituents, formulation properties or states, such as pH, an environment in which the formulation is placed, or a combination of formulation ingredients and an environment in which the formulation is placed. In one example, extended release can include release of a therapeutic agent at a level that is sufficient to provide a therapeutic effect or treatment for a non-immediate specified or intended duration of time.

As used herein, the term “modulate” refers to any change in biological state, i.e. increasing, decreasing, and the like.

As used herein, the units “parts per million,” “ppm,” “parts per billion,” “ppb,” and so on, when used to describe gas mixtures, are based on a volume basis. Accordingly, one part per million is equivalent to one part by volume out of a total of one million parts by volume.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, a composition that “increases” immune response provides a response level in a subject that is elevated as compared to a level at a previous point in time, such as a baseline level (e.g. prior to treatment), or as compared to an earlier treatment with a different (e.g. lower dose).

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 ml to about 80 ml” should also be understood to provide support for the range of “50 ml to 80 ml.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually, and further including decimal or fraction values such as 1.8, 2.3, 3.7, and 4.2.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, variants, modifications, and equivalents as may be included within the spirit and scope of the invention as defined by the appended claims.

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

The present disclosure describes methods and compositions that can be used to treat conditions that are responsive to NO treatment. The methods and compositions can be used to treat several specific indications. The indications include, but are not limited to, wounds, skin infections, fungal infections, respiratory infections, bacterial infections, viral infections, athlete's foot, onychomycosis, influenza, sinusitis, throat infections, acne, diabetic foot ulcers, warts, common cold, respiratory syncytial virus, corona virus, and others. In some examples, a NORS can be administered to provide NO gas that can kill and/or deactivate pathogens such as fungi, bacteria, and viruses. In further examples, the NO gas can provide additional therapeutic effects.

In certain examples, a method of treating a condition responsive to nitric oxide (NO) treatment in a subject can include administering a volume of a nitric oxide releasing solution (NORS) that provides a therapeutically effective amount of NO to an administration situs of the subject for a treatment duration. In other words, NO gas can contact the administration situs during the duration of the treatment. In some cases, the administration situs can be a location on or in the body of the subject. In certain examples, the administration situs can be an afflicted situs. In further examples, the administration situs can be a mucosal membrane such as an area of a subject's nasal passage, sinus, mouth, throat, lungs, or other locale in their respiratory system. In still further examples, the administration situs can be an area of skin of the subject, a foot of the subject, or a nail of the subject such as a toenail or fingernail.

Furthermore, the NORS can take any number of forms for administration depending on its intended use. In one embodiment, the NORS can be a spray such as a nasal spray, a sinus spray, a throat spray, a topical spray for use on skin, and so on. In another embodiment, the NORS can be a mouthwash or a solution for gargling. In a further embodiment, the NORS can be a sinus wash. In another embodiment, the NORS can be a liquid bath for immersing an administration situs, such as a foot bath. In yet another embodiment, the NORS can be soaked onto a cloth or substrate to be used as a wipe. In yet another embodiment, the NORS can be an aerosol. In further embodiments, the NORS can be a gel or cream. Nearly any form that is suitable for administration to a specifically designated situs can be used.

In some embodiments, the NORS can provide an extended release of gaseous nitric oxide (gNO) when administered to a situs. By “extended release,” it is meant that an effective amount of NO gas is released from the formulation at a controlled rate such that therapeutically beneficial levels (but below toxic levels) of gNO are maintained over an extended period of time. Specific examples of various NORS, including extended release NORS can be found in U.S. Pat. No. 9,730,956, which is incorporated herein by reference. An extended release NORS is beneficial in that the solution can be administered to a situs over a short period of time, while the release of NO from the solution continues following administration.

The NORS described herein can include a solution of a nitrite and an acid. In certain examples, the solution can become active when the nitrites and acids mix in saline or water in which the pH of the solution is below 4.0 and exhibits an increased or enhanced production level of nitric oxide gas over an extended period of time. In one embodiment, the pH of the active state of the nitric oxide releasing solution is between a pH of about 1.0 and a pH of about 4.0. In another embodiment, the pH of the active state of the nitric oxide releasing solution is between a pH of about 3.0 and a pH of about 4.0. In one embodiment, the pH is about 3.2. In another embodiment, the pH is about 3.6. In another embodiment, the pH is about 3.7. In one embodiment, the pH is about 4.0. In another embodiment, the pH is below about 4.0. Because the nitric oxide releasing solution of the present invention is not active until the acid interacts with the nitrites in liquid, the nitrite solution can be pre-made, transported and set up for administration while in its dormant state (pH greater than 4.0), without producing any appreciable nitric oxide gas or without losing its ability to produce an effective amount of nitric oxide gas. Then, when a user is ready to deliver or administer the solution for treatment of a human subject, the solution can be activated immediately prior to administration to the human subject by the addition of an acid (pH driven below 4.0), thereby maximizing the amount of nitric oxide gas produced by the administered dosage of solution.

In one embodiment, the pH of the solution can be lowered via addition of at least one acidifying agent into the solution. Introduction of the acidifying agent drives the solution reaction towards the reactants, thus reducing the pH (creating more acid), which in turn creates more nitric oxide gas.

For example, by introducing sodium nitrite (or other salts of nitrites) to a saline solution it will very slowly produce nitric oxide gas, but in an undetectable amount (as measured by chemiluminescence analysis methodology (ppb sensitivity)). The rate of NO produced from the solution increases as the pH is decreased, particularly as it drops below pH 4.0. NO is produced based on the following equilibrium equations:

NO₂⁻+H⁺→HNO₂ 1.

2HNO₂→N₂O₃+H₂O→H₂O+NO+NO₂ 2a.

3HNO₂⇄2NO+NO₃⁻+H₂O+H⁺ 2b.

Therefore, an acidifying agent, for example an acid, may donate the H⁺ to the nitrite (NO₂⁻). The more H⁺ present, the faster the reaction will go towards HNO₂ and the more NO will be produced.

In one embodiment, the nitric oxide releasing solution includes at least one nitric oxide releasing compound, such as nitrite, nitrite salt, or metal nitrite. In one embodiment, the solution is a saline-based solution. In one embodiment, the nitric oxide releasing compound is a nitrite, a salt thereof, and any combinations thereof. Non-limiting examples of nitrites include salts of nitrite such as sodium nitrite, potassium nitrite, magnesium nitrite, barium nitrite, and calcium nitrite, mixed salts of nitrite such as nitrite orotate, nitrite esters such as amyl nitrite, metal nitrites such as silver nitrite or copper nitrite. In one embodiment, the nitric oxide releasing compound is selected from the group consisting of sodium nitrite and potassium nitrite, and any combinations thereof. In another embodiment, the nitric oxide releasing compound is sodium nitrite. In one embodiment, the solution is comprised of sodium nitrite in a saline solution. In another embodiment, the solution is comprised of potassium nitrite in a saline solution.

In one embodiment, the concentration of nitrites in the solution can be from about 0.1% w/v to about 1.5% w/v. In another embodiment, the concentration of nitrites can be between 0.07% w/v and about 0.5% w/v. In one embodiment, the concentration of nitrites in the solution is no greater than about 0.5% w/v. In another embodiment, the concentration of nitrites in the solution is not greater than about 0.8% w/v. In another embodiment, the concentration of nitrites in the solution is not greater than about 1% w/v. In yet another embodiment, the concentration of nitrites in the solution is between about 0.07-0.5% w/v. In another embodiment, the concentration of nitrites in the solution is from about 0.2% to about 0.4% w/v. In still another embodiment, the concentration of nitrites in the solution is from about 0.4% to about 0.7% w/v. In another embodiment, the concentration of nitrites in the solution is from about 0.7% to about 0.9% w/v. In yet another embodiment, the concentration of nitrites in the solution is from about 0.9% to about 1.3% w/v. As used herein, the term “w/v” refers to the (weight of solute in grams/volume of solution in milliliters)×100%.

The solution of the present invention may also contain at least one acidifying agent. As described elsewhere here, the addition of at least one acidifying agent to the solution of the present invention contributes toward increased production of NO. Any acidifying agent which provides increased production of NO is contemplated by the present invention. In one embodiment, the acidifying agent is an acid. In some examples, the acid can be a weak acid. In other examples, the acid can be a strong acid. In further examples, the acid can include a mixture of a strong acid and a weak acid. In still further examples, the acid can be an organic acid. In other examples, the acid can be an inorganic acid. In further examples, the acid can include a combination of an organic acid and an inorganic acid. Non-limiting examples of acids include ascorbic acid, ascorbyl palmitate, salicylic acid, malic acid, lactic acid, citric acid, formic acid, benzoic acid, tartaric acid, hydrochloric acid, sulfuric acid, and phosphoric acid. In one embodiment, the acid is selected from the group consisting of ascorbic acid, citric acid, malic acid, hydrochloric acid, and sulfuric acid, and any combinations thereof. In one embodiment, the acid is citric acid.

As described above, the amount of acidifying agent present in the solution can affect the rate of the reaction to produce NO. In some embodiments, the amount of acidifying agent is from about 0.1% to about 4% w/v. In one embodiment, the amount of acidifying agent is no greater than about 0.5% w/v. In another embodiment, the amount of acidifying agent is about 0.5% w/v. In another embodiment, the amount of acidifying agent is about 0.2% w/v. In one embodiment, the amount of acidifying agent is about 0.07% w/v. In another embodiment, the amount of acidifying agent is between about 0.07-0.5% w/v. In yet another embodiment, the amount of acidifying agent is from about 0.2% to about 0.4% w/v. In another embodiment, the amount of acidifying agent is from about 0.4% to about 0.7% w/v. In another embodiment, the amount of acidifying agent is from about 0.7% to about 0.9%. In still another embodiment, the amount of acidifying agent is from about 0.9% to 1.3% w/v. In another embodiment, the amount of acidifying agent is from about 1.3% to about 2% w/v. In another embodiment, the amount of acidifying agent is from about 2% to about 3% w/v. In yet another embodiment, the amount of acidifying agent is from about 3% to about 4% w/v.

As mentioned above, NO gas can be unstable and have a relatively short half-life. Therefore, in some examples the NO gas may be available for a limited time after the NO is released from the NORS. In various examples, the NORS can be a two part composition. In certain examples, the NORS can be prepared just prior to administration to the situs through the administration of an acidifying agent to a dormant solution. For example, as described elsewhere herein, administration of the acidifying agent to the dormant solution results in the lowering of the pH of the dormant solution, thereby activating the nitric oxide releasing solution to be administered to the treatment site. The nitric oxide releasing solution provides for extended production of nitric oxide. In one embodiment, the nitric oxide releasing solution produces nitric oxide for a period of between 1 minute and 24 hours. In one embodiment, the nitric oxide releasing solution produces nitric oxide for a period of between 10 and 45 minutes. In one embodiment, the nitric oxide releasing solution produces nitric oxide for at least 15 minutes. In one embodiment, the nitric oxide releasing solution produces nitric oxide for at least 30 minutes. In another embodiment, the nitric oxide releasing solution produces nitric oxide for at least 1 hour. In another embodiment, the nitric oxide releasing solution produces nitric oxide for at least 4 hours. In another embodiment, the nitric oxide releasing solution produces nitric oxide for at least 8 hours. In another embodiment, the nitric oxide releasing solution produces nitric oxide for at least 12 hours. In another embodiment, the nitric oxide releasing solution produces nitric oxide for at least 24 hours. Thus, the administered nitric oxide releasing solution provides for continuous delivery of nitric oxide to the treatment site of the subject.

In certain examples, the rate at which the NORS produces NO can vary with time. For example, the NORS can be prepared by mixing an acidifying agent with a dormant solution, and the NORS can immediately begin to release NO gas at a particular rate. In some cases, the reaction that forms the NO gas can have an initial ramp-up period during which the rate at which NO is produced can increase up to a peak rate. After the peak rate, the rate of NO production can decrease over time until the reaction eventually is substantially finished. The initial rate of NO production, peak NO production, and time over which the rate of NO production falls can vary depending on the formulation of the NORS.

In some examples, the amount of NO produced by the NORS can be measured and quantified using methods and devices described in U.S. patent application Ser. No. 16/541,084, published as United States Patent Application Publication No. 2020/0110067, which is hereby incorporated herein by reference. In one example, NO produced by a NORS can be measured by releasing the NO from the NORS into a flow of carrier gas. The concentration of the NO in the carrier gas can then be quantified using an NO gas detector. Any suitable detector type can be used, such as chemiluminescence detectors, electrochemical detectors, fluorescence detectors, mass spectrometers, or others. In certain examples, the NO detector can be a chemiluminescence detector. Chemiluminescence is an analytical technique that generally involves the emission of light as a result of a chemical reaction. More specifically, decay of a molecule in an excited state to a lower energy state can cause an emission of light, which can be detected by a chemiluminescence analyzer/detector. As one example, nitric oxide (NO) can react with ozone (O₃) to produce excited NO that subsequently decays to a lower energy state and emits electromagnetic radiation that is photoelectrically detectable. In some examples, the detector can measure a concentration of NO gas in the carrier gas stream. The units of concentration can be, for example, parts-per-million (ppm) or parts-per-billion (ppb). In a certain example, the detector can be a Sievers™ Nitric Oxide Analyzer 280i or Zysense™ Nitric Oxide Analyzer 280i available from Zysense (USA).

Several parameters can be adjusted when measuring the NO released from the NORS. For example, the flow rate and pressure of the carrier gas can be adjusted. Increasing the flow rate of carrier gas, while the rate of NO production from the NORS remains constant, can result in a decreased concentration of the NO as measured by the detector because the NO is mixed with a larger volume of carrier gas. Conversely, decreasing the flow rate of the carrier gas can have the opposite effect. In some examples, the flow rate of carrier gas can be from about 0.025 liters per minute (L/min) to about 10 L/min. In certain examples, a standard flow rate can be selected so that the NO release rates of different NORS formulations can be compared to one another. In one example, the flow rate can be 1 L/min. In other examples, the flow rate can be 0.5 L/min, 2 L/min, 3 L/min, or 5 L/min.

Further, in some examples the carrier gas can be selected from gases that are inert with respect to NO. In some examples, the carrier gas can be free of or substantially free of oxygen or other reactive gas species. In further examples, the carrier gas can include nitrogen gas, a noble gas, helium gas, argon gas, neon gas, krypton gas, xenon gas, radon gas or combinations thereof.

In further examples, an amount of NO gas that is produced over time can be quantified by measuring the concentration of NO gas at various points in time, and then multiplying the concentration by the amount of time over which that concentration of NO was produced. At a given flow rate of carrier gas, the concentration of NO gas in the carrier gas can be proportional to the rate at which the NO gas is being produced from the NORS. For example, if a NORS produces a sufficient amount of NO gas that the concentration is 100 ppm, and the concentration remains steady at 100 ppm for 30 minutes, then the total amount of NO produced can be quantified by multiplying 100 ppm by 30 minutes to give 3,000 ppm*min. The units “ppm*min” can correspond to an actual amount of NO produced if the flow rate of the carrier gas is known.

In practice, a NORS formulation can often produce NO at a rate that is continuously changing. Therefore, it is not likely that a NORS would produce NO at a steady concentration of 100 ppm for 30 minutes. In order to accurately quantify the amount of NO gas being produced, the concentration of NO in the carrier gas can be measured at many points in time. These measurements can form a curve made up of concentration data points over time. By integrating the area under this curve (AUC), a total quantity of NO in the units of ppm*min can be determined. In other examples, the units used can be different, such as a different measure of concentration and a different measure of time. Other examples of units can include ppb*min or ppb*s or ppb*hr, for example. The AUC can be calculated by integrating the curve of concentration data points using a numerical integration method, such as multiplying each concentration data point by the time interval between data points, and taking the sum of the products. In various examples, the time interval between data points can be any suitable time period, for example from 1/1000^{th} of one second up to one minute or more. Smaller time intervals between measurements can yield a larger number of data points, which can, in some examples, provide a more accurate AUC. In certain examples, measurements can be made using a computer connected to the NO detector, and the computer can be programmed to calculate the AUC.

In certain examples, a standard procedure can be followed to measure the concentration of NO produced from a NORS. A standard method can also be used to calculate various quantities, such as the number of ppm*min of NO produced over a set period of time, or the peak NO concentration, or others. By using the same standard methods to quantify NO produced by different NORS formulations, the different NORS formulations can be compared one with another. In a particular example, a volume of a NORS can be introduced into a sample chamber. Any volume of NORS can be used. In some examples, the volume can be 1 mL, 2 mL, 3 mL, 5 mL, or another amount. The sample chamber can be made of a material that is inert with respect to NO in some examples. In other examples, the sample chamber can have an interior lining that is inert with respect to NO. A carrier gas can flow through the sample chamber at a fixed flow rate. In one example, the flow rate can be 1 L/min at standard temperature and pressure. In another example, the carrier gas can be nitrogen. The NORS can release NO into the flowing carrier gas, and the mixture of the carrier gas and NO gas can exit the sample chamber via an outlet. A chemiluminescence detector can be connected to the stream of carrier gas mixed with NO that is flowing from the outlet. The chemiluminescence detector can measure the concentration of NO in the gas mixture. The measurements can be taken at a specified time interval, such as once per minute, once per second, once per 1/10^{th} second, or another time interval. These measurements can be recorded as data points over a time period of 30 minutes. The AUC can be calculated from the data points. This quantity can be in units of ppm*min, and this can represent the total amount of NO gas that was released by the NORS sample over the course of 30 minutes. In further examples, the amount of NO released from a single mL of NORS can be calculated in units of ppm*min/mL. If the volume of NORS that was introduced into the sample chamber was 1 mL, then the AUC is the same value in ppm*min/mL. If a larger volume of NORS was used, such as 5 mL, then the AUC can be divided by the number of mL used to give the amount of NO released in ppm*min/mL.

Using the same measurement procedures, a peak NO concentration can be found by taking the maximum measurement value returned by the chemiluminescence detector. In some examples, the chemiluminescence detector can return measurement values in units of ppm. Accordingly, the peak NO concentration can be the highest instantaneous value in ppm that is measured during the 30 minutes.

In some examples, 1 mL of the NORS can release a desired amount of NO gas when measured over the course of 30 minutes, with a carrier gas flow rate of 1 L/min at a pressure of 1 atm as described above. In certain examples, the amount of NO released by the NORS can be from about 1 ppm*min/mL to about 500 ppm*min/mL. In other examples, the amount of NO can be from about 1 ppm*min/mL to about 100 ppm*min/mL, or from about 1 ppm*min/mL to about 60 ppm*min/mL, or from about 5 ppm*min/mL to about 100 ppm*min/mL, or from about 10 ppm*min/mL to about 100 ppm*min/mL, or from about 20 ppm*min/mL to about 100 ppm*min/mL. In further examples, the NORS can release a peak concentration of NO gas that is from about 0.1 ppm/mL to about 20 ppm/mL, when 1 mL of NORS is used to release NO gas into 1 L/min of carrier gas at 1 atm for 30 minutes, as described above. In particular examples, the peak concentration of NO released from 1 mL of NORS can be from about 0.3 ppm/mL to about 15 ppm/mL or from about 0.5 ppm/mL to about 12 ppm/mL.

The NORS may be administered in a variety of forms. The NORS may be administered as a liquid, a spray, a vapor, micro-droplets, mist, gargle, lavage, aerosol, gel, cream, or any form which provides the release of nitric oxide from the solution. In one embodiment, the NORS is administered as a spray. The amount or dosing volume of administered nitric oxide releasing solution may be varied in order to optimize the duration of nitric oxide production and delivery. In one embodiment, the amount of nitric oxide releasing solution administered is between about 0.1 mL and 5000 mL. In another embodiment, the amount of nitric oxide releasing solution administered is between about 10 mL and 1000 mL. In further embodiments, the amount of NORS administered can be from about 0.1 mL to about 1 mL, or from about 0.1 mL to about 2 mL, or from about 0.1 mL to about 5 mL, or from about 0.5 mL to about 1 mL, or from about 0.5 mL to about 2 mL, or from about 5 mL to about 500 mL, or from about 100 mL to about 300 mL, or from about 500 mL to about 5,000 mL. Certain types of NORS formulations can be administered in relatively large amounts, such as formulations that are used as a bath, wash, and so on. Other types of NORS formulations can be used in smaller volumes, such as gels or creams that are applied to small, localized areas, or nasal sprays. Some types of NORS formulations can be used in intermediate volumes, such as a mouthwash, sinus wash, and so on.

When an entire dose of NORS is measured by releasing NO from the NORS into a flow of carrier gas at 1 L/min and 1 atm for 30 minutes, as described above, the total amount of NO released can be quantified in units of ppm*min. In some examples, the amount of NO released from a dose of NORS can be from about 10 ppm*min to about 75,000 ppm*min. In further examples, the amount of NO can be from about 25,000 ppm*min to about 75,000 ppm*min, or from about 10 ppm*min to about 100 ppm*min, or from about 20 ppm*min to about 100 ppm*min, or from about 50 ppm*min to about 200 ppm*min, or from about 100 ppm*min to about 500 ppm*min, or from about 1,000 ppm*min to about 10,000 ppm*min, depending on the volume of the dose and the formulation of the NORS. In further examples, a dose of NORS can release a peak NO concentration during the 30 minute measurement period. The peak concentration can be from about 1 ppm to about 25,000 ppm, or from about 2 ppm to about 10 ppm, or from about 2 ppm to about 20 ppm, or from about 20 ppm to about 100 ppm, or from about 100 ppm to about 200 ppm, or from about 200 ppm to about 500 ppm, or from about 1,000 ppm to about 5,000 ppm, or from about 15,000 ppm to about 25,000 ppm.

In certain embodiments, nitric oxide releasing solution is directly administered into the upper respiratory tract of a subject. For example, in one embodiment, the nitric oxide releasing solution is sprayed into the upper respiratory tract of the subject. The solution may be administered into the upper respiratory tract of the subject once an hour, once a day, once a week, once every two weeks, once a month, once every two months, once a year, and any and all ranges therebetween as used to treat the subject. In one embodiment, the solution is sprayed once a week. In another embodiment, the solution is sprayed once a week for four consecutive weeks. The nitric oxide releasing solution provides for extended nitric oxide production, thereby providing continuous delivery of therapeutic nitric oxide to the upper respiratory infection of the subject.

The duration of administering the nitric oxide releasing solution to the subject may be varied in order to optimize delivery. In one embodiment, the nitric oxide releasing solution is administered over a time period of less than 5 seconds. In another embodiment, the nitric oxide releasing solution is administered over a time period of about 5 seconds. In another embodiment, the nitric oxide releasing solution is administered over a time period of about 30 seconds. In another embodiment, the nitric oxide releasing solution is administered over a time period of about 1 minute to about 20 minutes. The nitric oxide releasing solution can also be administered according to a dosage regimen that can include a single dose or multiple doses given at specific times. In some examples, doses can be administered at a frequency from about once per day to about 5 times per day, or from about once per day to about once every 50 days, or from about once per day to about once per week.

It should be noted that the time period for administering a dose of NORS to a subject can be different from the time period used to measure the concentration of NO gas released from the NORS in order to characterize the NORS formulation. As mentioned above, in some examples a standard procedure can be used to characterize a NORS formulation and to quantify the amount of NO gas released by the NORS. The standard procedure may call for measuring NO release over a time period, such as 30 minutes. However, when administering the NORS formulation to a subject, the time for administering a dose of NORS can be different. Similarly, the volume of NORS administered to a subject can be different from the volume used in the measurement procedure for quantifying the amount of NO released from the NORS. Accordingly, the amount of NO that is actually delivered to a subject can be different from the amount that is released during the standard measurement procedure.

Treatment of a respiratory disease by way of the present invention comprises the delivery of a nitric oxide releasing solution into the upper respiratory tract of the subject to be treated. For example, in certain embodiments, the nitric oxide releasing solution may be injected, sprayed, inhaled, or instilled into the respiratory tract of the subject. The nitric oxide releasing solution may be administered to the respiratory tract of the subject via the nasal cavity or oral cavity of the subject. In one embodiment, the nitric oxide releasing solution is sprayed into the upper respiratory tract of the subject. In one embodiment, the solution is administered to the subject intranasally. In one embodiment, the solution is administered to the sinuses. The nitric oxide releasing solution provides for extended nitric oxide production, thereby providing continuous delivery of therapeutic nitric oxide to the upper respiratory tract of the subject.

In other examples, the skin of a subject can be treated by applying a nitric oxide releasing solution to an area of the skin. The nitric oxide releasing solution can be in the form of a liquid bath, a spray, a cream, a gel, or another form that can be applied to the skin. In certain examples, the skin of the foot can be treated by applying the nitric oxide releasing solution to the skin of the foot. In further examples, fingernails or toenails and the surrounding skin can be treated by applying the nitric oxide releasing solution to a fingernail, toenail, or the surrounding skin. In various examples, a specific dosage of nitric oxide can be provided to a specific administration situs. The nitric oxide can be provided by a nitric oxide releasing solution that is formulated to provide this specific dosage of nitric oxide.

In one example, a NORS can be used to treat athlete's foot. Athlete's foot can be treated by administering a NORS that is formulated to provide a therapeutically effective dose of NO for treating athlete's foot. In some examples, the NO can be provided to an administration situs that is at a foot of a subject. In a particular example, the NORS can be in the form of a liquid solution. The liquid solution can be used as a foot bath. The foot of the subject can be dipped or immersed in the foot bath for a treatment duration.

In certain examples, the NORS used to treat athlete's foot can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 25,000 ppm*min to about 75,000 ppm*min. In further examples, the amount of NO released from a single mL of the NORS can be from about 5 ppm*min/mL to about 15 ppm*min/mL. In still further examples, the volume of a dose of the NORS can be from about 500 mL to about 10,000 mL. In further examples, the dose of NORS can release a peak NO concentration of from about 5,000 ppm to about 20,000 ppm.

The NORS can be formulated to provide a therapeutic amount of NO for treating athlete's foot. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating athlete's foot can include administering the NORS according to a dosage regimen of soaking the foot in a foot bath containing the NORS for 30 minutes. In further examples, the dosage regimen can include soaking the foot in the foot bath once every 4 days.

In another example, a NORS can be used to treat onychomycosis. Onychomycosis can be treated by administering a NORS that is formulated to provide a therapeutically effective dose of NO for treating onychomycosis. In some examples, the NO can be provided to an administration situs that is at a toenail or fingernail of a subject. In a particular example, the NORS can be in the form of a liquid solution, a gel, or a cream. The liquid solution, gel, or cream, can be applied to a nail of a subject for a treatment duration.

In certain examples, the NORS used to treat onychomycosis can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 25 ppm*min to about 75 ppm*min. In further examples, the amount of NO release from a single mL of the NORS can be from about 25 ppm*min/mL to about 75 ppm*min/mL. In still further examples, the volume of a dose of the NORS can be from about 0.1 mL to about 2 mL. In further examples, the dose of NORS can release a peak NO concentration of from about 5 ppm to about 20 ppm.

The NORS can be formulated to provide a therapeutic amount of NO for treating onychomycosis. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating onychomycosis can include administering the NORS according to a dosage regimen of applying the NORS to the nail daily for a period of 5 to 30 days.

In another example, a NORS can be used to treat influenza. Influenza can be treated by administering a NORS that is formulated to provide a therapeutically effective dose of NO for treating influenza. In some examples, the NO can be provided to an administration situs that is in a nasal cavity of a subject. In a particular example, the NORS can be in the form of a nasal spray. The nasal spray can be applied to a nasal cavity a subject for a treatment duration.

In certain examples, the NORS used to treat influenza can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 1 ppm*min to about 15 ppm*min or from about 5 ppm*min to about 10 ppm*min. In further examples, the amount of NO release from a single mL of the NORS can be from about 2 ppm*min/mL to about 30 ppm*min/mL. In still further examples, the volume of a dose of the NORS can be from about 0.1 mL to about 2 mL or from about 0.1 mL to about 1 mL. In further examples, the dose of NORS can release a peak NO concentration of from about 0.5 ppm to about 5 ppm.

The NORS can be formulated to provide a therapeutic amount of NO for treating influenza. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating influenza can include administering the NORS according to a dosage regimen of administering nasal spray as needed.

In another example, a NORS can be used to treat a respiratory viral infection. Respiratory viral infections, such as common cold, respiratory syncytial virus, and corona virus, can be treated by administering a NORS that is formulated to provide a therapeutically effective dose of NO for treating the respiratory viral infection. In some examples, the NO can be provided to an administration situs that is in a nasal cavity of a subject. In a particular example, the NORS can be in the form of a nasal spray. The nasal spray can be applied to a nasal cavity a subject for a treatment duration.

In certain examples, the NORS used to treat a respiratory viral infection can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 1 ppm*min to about 15 ppm*min or from about 5 ppm*min to about 10 ppm*min. In further examples, the amount of NO released from a single mL of the NORS can be from about 2 ppm*min/mL to about 30 ppm*min/mL. In still further examples, the volume of a dose of the NORS can be from about 0.1 mL to about 2 mL or from about 0.1 mL to about 1 mL. In further examples, the dose of NORS can release a peak NO concentration of from about 0.5 ppm to about 5 ppm.

The NORS can be formulated to provide a therapeutic amount of NO for treating a respiratory viral infection. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating a respiratory viral infection can include administering the NORS according to a dosage regimen of administering nasal spray as needed.

In another example, a NORS can be used to treat sinusitis. Sinusitis can be treated by administering a NORS that is formulated to provide a therapeutically effective dose of NO for treating sinusitis. In some examples, the NO can be provided to an administration situs that is in a sinus cavity of a subject. In a particular example, the NORS can be in the form of a liquid lavage or irrigation solution. In certain examples, the liquid lavage or irrigation solution can have a volume of about 240 mL or less.

In certain examples, the NORS used to treat sinusitis can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 100 ppm*min to about 5,000 ppm*min, or from about 100 ppm*min to about 500 ppm*min, or from about 500 ppm*min to about 2,000 ppm*min, or from about 2,000 to about 5,000 ppm*min. In further examples, the amount of NO release from a single mL of the NORS can be from about 0.5 ppm*min/mL to about 30 ppm*min/mL, or from about 1 ppm*min/mL to about 4 ppm*min/mL, or from about 4 ppm*min/mL to about 10 ppm*min/mL, or from about 10 ppm*min/mL to about 30 ppm*min/mL. In still further examples, the volume of a dose of the NORS can be from about 150 mL to about 300 mL. In further examples, the dose of NORS can release a peak NO concentration of from about 50 ppm to about 1,500 ppm.

The NORS can be formulated to provide a therapeutic amount of NO for treating sinusitis. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating influenza can include administering the NORS according to a dosage regimen of administering the NORS daily for a period of from about 10 days to about 50 days.

In another example, a NORS can be used to treat a throat infection. The throat infection can be treated by administering a NORS that is formulated to provide a therapeutically effective dose of NO for treating the throat infection. In some examples, the NO can be provided to an administration situs that is the throat of a subject. In a particular example, the NORS can be in the form of a gargling liquid solution. The solution can be gargled by the subject for a treatment duration.

In certain examples, the NORS used to treat the throat infection can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 200 ppm*min to about 800 ppm*min or from about 400 ppm*min to about 600 ppm*min. In further examples, the amount of NO release from a single mL of the NORS can be from about 10 ppm*min/mL to about 40 ppm*min/mL. In still further examples, the volume of a dose of the NORS can be from about 5 mL to about 40 mL or from about 10 mL to about 30 mL. In further examples, the dose of NORS can release a peak NO concentration of from about 100 ppm to about 200 ppm.

The NORS can be formulated to provide a therapeutic amount of NO for treating a throat infection. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating a throat infection can include administering the NORS according to a dosage regimen of administering the NORS from 1 to 3 times per day for a period of about 1 to about 5 days.

In another example, a NORS can be used to treat acne. Acne can be treated by administering a NORS that is formulated to provide a therapeutically effective dose of NO for treating acne. In some examples, the NO can be provided to an administration situs that is at a skin area of a subject. In a particular example, the NORS can be in the form of a gel or cream. The NORS can be applied to the skin of the subject for a treatment duration.

In certain examples, the NORS used to treat acne can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 10 ppm*min to about 60 ppm*min or from about 20 ppm*min to about 60 ppm*min. In further examples, the amount of NO release from a single mL of the NORS can be from about 5 ppm*min/mL to about 30 ppm*min/mL. In still further examples, the volume of a dose of the NORS can be from about 1 mL to about 5 mL. In further examples, the dose of NORS can release a peak NO concentration of from about 5 ppm to about 20 ppm.

The NORS can be formulated to provide a therapeutic amount of NO for treating acne. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating acne can include administering the NORS according to a dosage regimen of applying the NORS to the skin from 1 to 3 times per day for a period of about 10 days to about 50 days.

In another example, a NORS can be used to treat a diabetic foot ulcer. Diabetic foot ulcer can be treated by administering a NORS that is formulated to provide a therapeutically effective dose of NO for treating diabetic foot ulcer. In some examples, the NO can be provided to an administration situs that is at a foot of a subject. In a particular example, the NORS can be in the form of a liquid solution in a foot bath or a gel. The NORS can be applied to the foot of the subject for a treatment duration.

In certain examples, the NORS used to treat diabetic foot ulcer can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 100 ppm*min to about 75,000 ppm*min or from about 100 ppm*min to about 400 ppm*min or from about 25,000 ppm*min to about 75,000 ppm*min. In further examples, the amount of NO release from a single mL of the NORS can be from about 5 ppm*min/mL to about 80 ppm*min/mL or from about 10 ppm*min/mL to about 60 ppm*min/mL. In still further examples, the volume of a dose of the NORS can be from about 5 mL to about 10,000 mL. In further examples, the dose of NORS can release a peak NO concentration of from about 50 ppm to about 20,000 ppm.

The NORS can be formulated to provide a therapeutic amount of NO for treating diabetic foot ulcer. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating diabetic foot ulcer can include administering the NORS according to a dosage regimen of applying the NORS to the foot of the subject from 1 to 3 times per day for a period of about 10 days to about 30 days.

In another example, a NORS can be used to treat warts. Warts can be treated by administering a NORS that is formulated to provide a therapeutically effective dose of NO for treating warts. In some examples, the NO can be provided to an administration situs that is at a skin area of a subject. In a particular example, the NORS can be in the form of a gel or a liquid solution. The NORS can be applied to the skin of the subject for a treatment duration.

In certain examples, the NORS used to treat warts can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 20 ppm*min to about 400 ppm*min or from about 20 ppm*min to about 80 ppm*min or from about 200 ppm*min to about 400 ppm*min. In further examples, the amount of NO release from a single mL of the NORS can be from about 10 ppm*min/mL to about 60 ppm*min/mL. In still further examples, the volume of a dose of the NORS can be from about 0.5 mL to about 10 mL. In further examples, the dose of NORS can release a peak NO concentration of from about 1 ppm to about 60 ppm.

The NORS can be formulated to provide a therapeutic amount of NO for treating warts. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of treating warts can include administering the NORS according to a dosage regimen of applying the NORS to the skin from 1 to 3 times per day for a period of about 1 day to about 10 days.

In another example, a NORS can be used to disinfect the skin of a subject. The skin can be disinfected by administering a NORS that is formulated to provide a therapeutically effective dose of NO for disinfecting skin. In some examples, the NO can be provided to an administration situs that is at a skin area of a subject. In a particular example, the NORS can be in the form of a gel or cream. The NORS can be applied to the skin of the subject for a treatment duration.

In certain examples, the NORS used to disinfect the skin can release a specific amount of NO gas when measured by releasing the NO gas into a flow of inert carrier gas at a flow rate of 1 L/min and a pressure of 1 atm for a period of 30 minutes. The concentration of the NO in the carrier gas can be measured periodically using a chemiluminescence detector that measures the concentration in units of ppm. The total amount of NO released during the 30 minutes can be found by calculating the area under the curve (AUC) of the NO concentration measurements. In some examples, the amount of NO released from a dose of the NORS can be from about 450 ppm*min to about 750 ppm*min. In further examples, the amount of NO release from a single mL of the NORS can be from about 5 ppm*min/mL to about 25 ppm*min/mL. In still further examples, the volume of a dose of the NORS can be from about 50 mL to about 20 mL. In further examples, the dose of NORS can release a peak NO concentration of from about 1 ppm to about 20 ppm.

The NORS can be formulated to provide a therapeutic amount of NO for disinfecting the skin. In certain examples, the NORS can include a nitrite and an acidifying agent. In some examples, the NORS can include a nitrite at a concentration of from about 0.1 g/L to about 20 g/L, in terms of grams of the nitrite in one liter of the NORS. In further examples, the NORS can include the acidifying agent at a concentration of from about 0.1 g/L to about 60 g/L.

In certain examples, methods of disinfecting the skin can include administering the NORS according to a dosage regimen of applying the NORS to the skin from 1 to 3 times per day for a period of about 10 days to about 20 days.

As mentioned above, in various examples, the NORS can be prepared in a variety of forms depending on the specific indication and method of treatment. Some forms of NORS that can be prepared include liquid solutions such as solutions in water and solutions in saline, gargling solutions, liquid baths, liquid lavages, liquid rinses, sprays such as nasal sprays or throat sprays, sinus washes, gels, creams, foams, ointments, lotions, pastes, emulsions, aerosols, and others. In the various forms, the NORS compositions can include a combination of the ingredients described above. In certain examples, the NORS can include a nitrite, and acidifying agent, water, and optionally additional ingredients such as inactive ingredients. The nitrite and acidifying agent can be included in amounts as needed to produce a desired amount of NO gas. Various inactive ingredients can include stabilizing agents, carriers, preservatives, emollients, adjuvants, and others.

The NORS can be prepared as a liquid solution in some examples. As mentioned above, liquid solutions can have a variety of specific forms, such as solutions in water, solutions in saline, gargling solutions, liquid baths, liquid lavages, liquid rinses, sprays, sinus washes, and others. In certain examples, a NORS can be prepared by mixing a nitrite and an acidifying agent in water. The nitrite and acidifying agent can be included in an amount as needed to produce a desired amount of NO gas. Examples of nitrites and acidifying agents, as well as example amounts of these ingredients, are described above. In some examples, a NORS in the form of a liquid solution can also include a stabilizing agent, a carrier, a preservative, an emollient, an adjuvant, or a combination thereof.

In examples that include a stabilizing agent, the stabilizing agent can be included in an amount from about 0.1 wt % to about 50 wt % with respect to the total weight of the NORS. In further examples, the amount of stabilizing agent can be from about 1 wt % to about 50 wt %, or from about 1 wt % to about 30 wt %, or from about 1 wt % to about 15 wt %, or from about 1 wt % to about 8 wt %. In certain examples, the stabilizing agent can include an oxygen scavenger, a lipid, a synthetic polymer, a natural polymer, a co-polymer, or a combination thereof.

In further examples, the NORS in the form of a liquid solution can include a carrier, which can include water, including deionized and/or deoxygenated water, a surfactant, a polymer, or combinations thereof. Surfactants can include lipophilic surfactants and hydrophilic surfactants. In some examples, lipophilic surfactants can include mono-, di-glycerides of fatty acids like glyceryl monolinoleate (e.g. Maisine® 35-1), mono- and di glycerides of caprylic, capric acid (e.g. Capmul® MCM), glyceryl monooleate, reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (e.g. Labrafil® M 2125 CS), PEG-6 almond oil (e.g. Labrafil® M 1966 CS), PEG-6 apricot kernel oil (e.g. Labrafil® M 1944 CS), PEG-6 olive oil (e.g. Labrafil® M 1980 CS), PEG-6 peanut oil (e.g. Labrafil® M 1969 CS), PEG-6 hydrogenated palm kernel oil (e.g. Labrafil®. M 2130 BS), PEG-6 palm kernel oil (e.g. Labrafil® M 2130 CS), PEG-6 triolein (e.g. Labrafil® M 2735 CS), PEG-8 corn oil (e.g. Labrafil® WL 2609 BS), PEG-20 corn glycerides (e.g. Crovol® M40), PEG-20 almond glycerides (e.g. Crovol® A40), lipophilic polyoxyethylenepolyoxypropylene block co-polymers (e.g. Pluronic® L92, L101, L121 etc.); propylene glycol fatty acid esters, such as propylene glycol monolaurate (e.g. Lauroglycol FCC), propylene glycol ricinoleate (e.g. Propymuls), propylene glycol monooleate (e.g. Myverol P-06), propylene glycol dicaprylate/dicaprate (e.g. Captex® 200), and propylene glycol dioctanoate (e.g. Captex® 800), propylene glycol mono-caprylate (e.g. Capryol® 90); propylene glycol oleate (e.g. Lutrol OP2000); propylene glycol myristate; propylene glycol mono stearate; propylene glycol hydroxy stearate; propylene glycol ricinoleate; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g. Arlacel® 186); sterol and sterol derivatives such as cholesterol, sitosterol, phytosterol, phytosterol fatty acid esters, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (e.g. Arlacel 20), sorbitan monopalmitate (e.g. Span-40), sorbitan monooleate (e.g. Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, myristic acid, menthol, menthol derivatives, lecithin, phosphatidyl choline, bile salts, and the like, and mixtures thereof.

In other examples, the carrier can include a hydrophilic surfactant. Example hydrophilic surfactants can include non-ionic surfactants, ionic surfactants and zwitterionic surfactants. Specifically the hydrophilic surfactants include, but are not limited to, alcohol-oil transesterification products; polyoxyethylene hydrogenated vegetable oils; polyoxyethylene vegetable oils; alkyl sulphate salts, dioctyl sulfosuccinate salts; polyethylene glycol fatty acids esters; polyethylene glycol fatty acids mono- and di-ester mixtures; polysorbates, polyethylene glycol derivatives of tocopherol and the like. It should be noted that the combinations of two or more hydrophilic surfactants from the same or different classes are within the scope of this invention and are together can be referred to as the hydrophilic surfactant unless explicitly specified. Further examples of hydrophilic surfactants can include PEG-8 caprylic/capric glycerides, lauroyl macrogol-32 glyceride, stearoyl macrogol glyceride, PEG-40 hydrogenated castor oil, PEG-35 castor oil, sodium lauryl sulfate, sodium dioctyl sulfosuccinate, polyethylene glycol fatty acids mono- and di-ester mixtures, polysorbate 80, polysorbate 20, polyethylene glycol 1000 tocopherol succinate, phytosterols, phytosterol fatty acid esters, and mixtures thereof.

In some embodiments, surfactants can include sterols and derivatives of sterols. In various embodiments, these surfactants can be hydrophilic or lipophilic. Examples of hydrophilic sterol surfactants are lanosterol PEG-24 cholesterol ether (e.g. Solulan C-24, Amerchol), PEG-30 soya sterol (e.g. Nikkol BPS-30, from Nikko), PEG-25 phyto sterol (e.g. Nikkol BPSH-25 from Nikko), PEG-30 cholestanol (e.g. Nikkol DHC, from Nikko). Examples of Lipophilic Sterol Surfactants are Cholesterol, sitosterol, Phytosterol (e.g. GENEROL series from Henkel), PEG-5 soya sterol (e.g. Nikkol BPS-S, from Nikko), PEG-10 soya sterol (e.g. Nikkol BPS-10 from Nikko), PEG-20 soya sterol (e.g. Nikkol BPS-20 from Nikko).

In some examples, the surfactant can comprise from about 1 wt % to about 20 wt % of the carrier. In other examples, the surfactant can be at least 20 wt % of the carrier. In further examples, the surfactant can comprises from about 1 wt % to about 10 wt % of the carrier or from about 1 wt % to about 5 wt % of the carrier.

In further examples, the NORS liquid solution composition can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

The NORS liquid solution can also include an emollient in some examples. Non-limiting examples of emollients can include, but are not limited to, aloe vera, lanolin, urea, petrolatum, shea butter, cocoa butter, mineral oil, paraffin, beeswax, squalene, jojoba oil, coconut oil, sesame oil, almond oil, cetyl alcohol, stearyl alcohol, olive oil, oleic acid, triethylhexanoin, glycerol, sorbitol, propylene glycol, cyclomethicone, dimethicone, the like, or combinations thereof. In certain examples, the emollient can be included in an amount from about 0.1 wt % to about 20 wt % with respect to the total weight of the NORS liquid solution. In further examples, the emollient can be included in an amount from about 0.1 wt % to about 10 wt % or from about 0.1 wt % to about 5 wt %.

In further examples, the NORS liquid solution can also include an adjuvant. Some examples of adjuvants can include analgesic adjuvants, aluminum hydroxide, aluminum phosphate, calcium phosphate hydroxide, paraffin oil, detergents, plant saponins, cytokines, and combinations thereof. In certain examples, the adjuvant can be included in an amount from about 0.1 wt % to about 20 wt % with respect to the total weight of the NORS liquid solution. In further examples, the adjuvant can be included in an amount from about 0.1 wt % to about 10 wt % or from about 0.1 wt % to about 5 wt %.

In other examples, the NORS composition can be in the form of a gel, cream, ointment, lotion, liniment, or paste. As with the NORS liquid solution composition described above, these formulations can also include a nitrite and an acidifying agent in amounts as needed to produce a desired amount of NO gas. Additionally, these NORS formulations can also include a stabilizing agent, a carrier, a preservative, an emollient, an adjuvant, or a combination thereof. These ingredients can include the examples described above, in amounts as described above.

In further examples, a NORS in the form of a gel, cream, ointment, lotion, liniment, or paste can also include a thickener, a penetration enhancer, or a combination thereof. In some examples, thickeners can include polymers having thickening properties. Non-limiting examples of a polymer having thickening properties can include, but are not limited to, a hydrophobically modified cross-linked acrylate copolymer (Carbopol® Ultrez 20). Other polymers having similar properties may also be used. Non-limiting examples of polymers having thickening properties can include PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C10-C30 alkyl acrylate crosspolymers, and combinations thereof. Other example thickeners can include polyacrylic acids (e.g. Carbopols, for example), gelatin, pectin, tragacanth, methyl cellulose, hydroxylethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, the like, or combinations thereof. In some examples, the thickener can be included in an amount from about 1 wt % to about 20 wt %, or from about 1 wt % to about 10 wt %, or from about 5 wt % to about 15 wt %, based on the total weight of the NORS composition.

In other examples, the NORS can include a penetration enhancer. Non-limiting examples of a penetration enhancer can include, but are not limited to, ethanol, propylene glycol, oleic acid and other fatty acids, azone, terpenes, terpenoids, bile acids, isopropyl myristate and other fatty esters, dimethyl sulphoxides, N-methyl-2-pyrrolidone and other pyrrolidones, the like, or combinations thereof. In certain examples, the penetration enhancer can be included in an amount from about 0.1 wt % to about 5 wt %, or from about 0.1 wt % to about 3 wt %, or from about 0.1 wt % to about 2 wt %, based on the total weight of the NORS composition.

In further examples, the NORS composition can also include fumed silica, mica, talc, titanium dioxide, kaolin, aluminum glycinate, ethylenediaminetetraacetic acid, fragrances, colorants, or combinations thereof. These additional ingredients can be included in an amount from about 1 wt % to about 20 wt %, or from about 1 wt % to about 10 wt %, or from about 1 wt % to about 5 wt %, in some examples.

### EXPERIMENTAL EXAMPLES

### Example 1: NO Release Measurements

A series of example nitric oxide releasing solution formulations was prepared. The individual formulations were formulated to treat specific conditions. The conditions to be treated included: athlete's foot, onychomycosis, influenza, respiratory viral infection such as the common cold, sinusitis, throat infection, acne, diabetic foot ulcer, and warts. An example NORS was also prepared that was formulated for skin disinfection.

The examples NORS formulations were tested for NO release by measuring the amount of NO gas released from the formulations. The following procedure was used to measure the NO gas. 5 mL of a NORS formulation was introduced into a sample chamber having an interior surface that was inert with respect to NO. Nitrogen was used as a carrier gas. The nitrogen flowed into an inlet in the sample chamber and out through an outlet at a flow rate of 1 L/min. The outlet led to a chemiluminescence detector that measured the concentration of NO in the gas stream in units of ppm. The chemiluminescence detector provided the concentration measurement to a computer that was programmed to record concentration measurements at regular time intervals over a period of 30 minutes. The period of 30 minutes began when the NORS sample was introduced into the sample chamber. The computer was also programmed to calculate the area under the curve (AUC) of the concentration data points to find the total amount of NO that was released in units of ppm*min. This value was then divided by the volume of 5 mL to find the amount of NO released from a single mL of the NORS, in units of ppm*min/mL. The largest ppm concentration measurement was also selected as the peak NO concentration that occurred during the 30 minute period. Dividing the peak NO concentration measured by 5 mL yields the peak NO concentration per 1 mL of the NORS formulation. The calculations of “per mL” values are based on the assumption that the amount of NO released is proportional to the volume of NORS in the sample chamber.

Table 1 shows the amounts of NO released by the example NORS formulations based on a single mL of the NORS and based on a full dose of the NORS, along with example dose volumes and peak NO concentrations.

TABLE 1

| | | Peak NO | | |
|---|---|---|---|---|
| | NO Released | Concentration | NO Released | Dose |
| | per 1 mL | per 1 mL | per Dose | Volume |
| Sample | (ppm*min/mL) | (ppm/mL) | (ppm*min) | (mL) |
| Athlete's Foot | 12.85 | 3.514 | 64230 | 5000 |
| Onychomy cosis | 52.00 | 11.034 | 52 | 1 |
| Influenza | 13.23 | 4.268 | 6.616 | 0.5 |
| Resp. Viral | 13.23 | 4.268 | 6.616 | 0.5 |
| Infection | | | | |
| Sinusitis A | 1.91 | 0.6 | 458.4 | 240 |
| Sinusitis B | 5.46 | 1.52 | 1310.4 | 240 |
| Sinusitis C | 13.23 | 4.268 | 3175.7 | 240 |
| Throat Infection | 23.35 | 7.12 | 466.9 | 20 |
| Acne A | 13.23 | 4.268 | 26.46 | 2 |
| Acne B | 23.35 | 7.12 | 46.69 | 2 |
| Diab. Foot Ulcer A | 12.85 | 3.514 | 64230 | 5000 |
| Diab. Foot Ulcer B | 52.00 | 11.034 | 260 | 5 |
| Warts A | 13.23 | 4.3 | 66.2 | 5 |
| Warts B | 52.00 | 11.0 | 260 | 5 |
| Skin Disinfection A | 13.23 | 4.3 | 520 | 100 |
| Skin Disinfection B | 23.35 | 7.1 | 660 | 100 |

### Example 2: Exposure, Safety and Efficacy of NONS

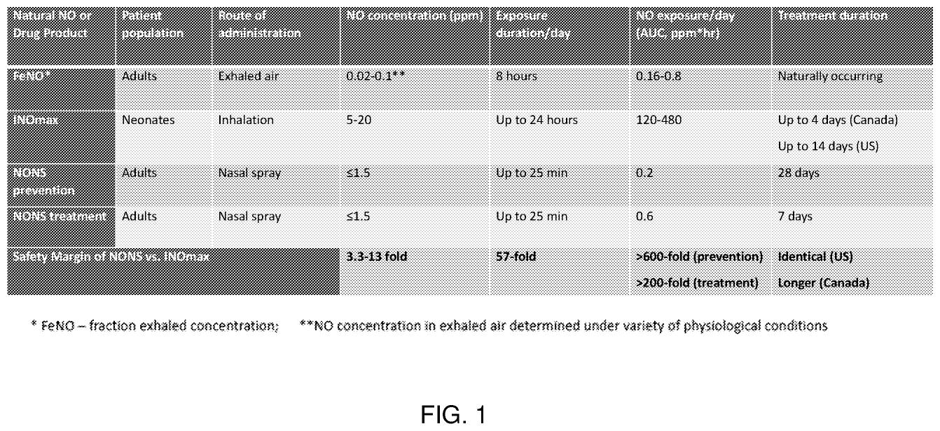

As depicted in the Table in FIG. 1, nonclinical pharmacokinetics (PK) and toxicology testing was conducted for NO by measuring NONS prevention and NONS treatment compared to natural NO and INOmax.

The NO concentration in exhaled air determined under a variety of physiological conditions was measured. The patient population included adults with a route of administration including exhaled air. The NO concentration was measured to range from 0.02 ppm to 0.1 ppm. The exposure duration per day was approximately 8 hours per day because exhaled time is about ⅓ of total breathing time. The NO exposure per day as calculated using area under the curve (AUC) in units of ppm*hr was calculated to be in a range of from 0.16 to 0.8 ppm*hr. This calculation of daily exposure was based on 0.02 ppm for a duration of 8 hours at the lower end of the range and 0.1 ppm for a duration of 8 hours at the upper end of the range. Because the exhaled air from the patient population was naturally occurring, the level of exposure and the level of safety provides a baseline for comparing exposure, safety, and efficacy of various treatments.

INOmax is a treatment that has been administered to a patient population of neonates, with a route of administration that is inhalation. The NO is administered as a gas at concentration ranges of from 5 to 20 ppm. The exposure time can be up to 24 hours. Based on the administered range of 5 to 20 ppm and the exposure time of 24 hours, the amount of NO exposure per day as calculated using AUC in units of ppm*hr was calculated to be in a range of from 120 ppm*hr to 480 ppm*hr. The treatment duration for INOmax can be up to 4 days in Canada or up to 14 days in the US.

NONS (e,g, NORS provided as a nasal spray) can be used as a preventive treatment as a nasal spray that can be administered to adults for a duration of 28 days. The amount of NO concentration administered can be a maximum NO peak concentration of up to 1.5 ppm for each 0.5 mL volume of NONS. The duration of exposure can be up to 30 minutes based on 6 treatments of 5 minutes each using a volume of 0.5 mL for each treatment. The maximum total exposure for 5 mL of NONS was measured to be 54 ppm*min, which can be calculated to be 5.4 ppm*min for a 0.5 mL treatment volume. Thus, for each treatment of 5 minutes, the maximum total exposure was 5.4 ppm*min for a 0.5 mL treatment volume, which can be calculated to be 0.1 ppm*hr by dividing 5.4 ppm*min by 60 min/hr. For a maximum exposure time of 5 minutes per treatment and 2 treatments in one day, the total time of exposure is 10 minutes per day. With each treatment providing 0.1 ppm*hr of NO exposure, then 2 treatments in one day is 0.2 ppm*hr of NO exposure. Therefore, based on a concentration administered of 1.5 ppm and an exposure time of 10 minutes, the NO exposure per day as calculated using AUC in units of ppm*hr was calculated to be 0.2 ppm*hr.

NONS can be used as a nasal spray that can be administered to adults in 6 separate treatments per day for a duration of 7 days. Thus, as calculated in the preceding, for each treatment of 5 minutes, the maximum total exposure was 5.4 ppm*min for a 0.5 mL treatment volume, which can be calculated to be 0.1 ppm*hr by dividing 5.4 ppm*min by 60 min/hr. For a maximum exposure time of 5 minutes per treatment and 6 treatments in one day, the total time of exposure is 30 minutes per day. With each treatment providing 0.1 ppm*hr of NO exposure, then 6 treatments in one day is 0.6 ppm*hr of NO exposure. Therefore, based on a concentration administered of 1.5 ppm and an exposure time of 30 minutes, the NO exposure per day as calculated using AUC in units of ppm*hr was calculated to be 0.6 ppm*hr.

As depicted in the table in FIG. 1, the safety margin of NONS prevention and NONS treatment compared to INOmax was calculated to be 3.3 to 13-fold safer for NONS prevention and NONS treatment as measured by NO concentration administered in ppm. The safety margin of NONS prevention and NONS treatment compared to INOmax was calculated to be 57-fold safer for NONS prevention and NONS treatment as measured by NO exposure duration per day. The safety margin of NONS prevention and NONS treatment compared to INOmax was calculated to be about 600-fold safer for NONS prevention and 200-fold safer for NONS treatment as measured by NO exposure per day as calculated using AUC in units of ppm*hr. The treatment duration for NONS prevention and NONS treatment compared to INOmax can be shorter in some cases and longer in some cases.

### Example 3a: Porcine Infected Wounds

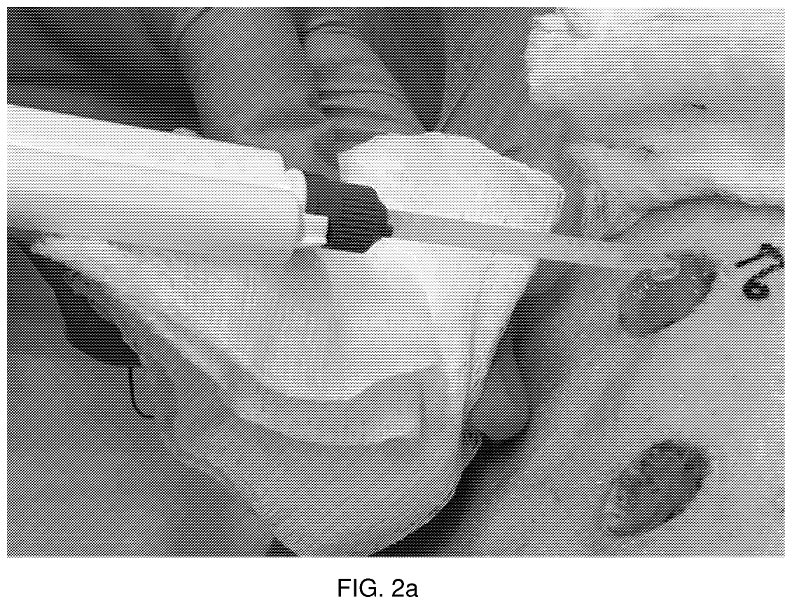

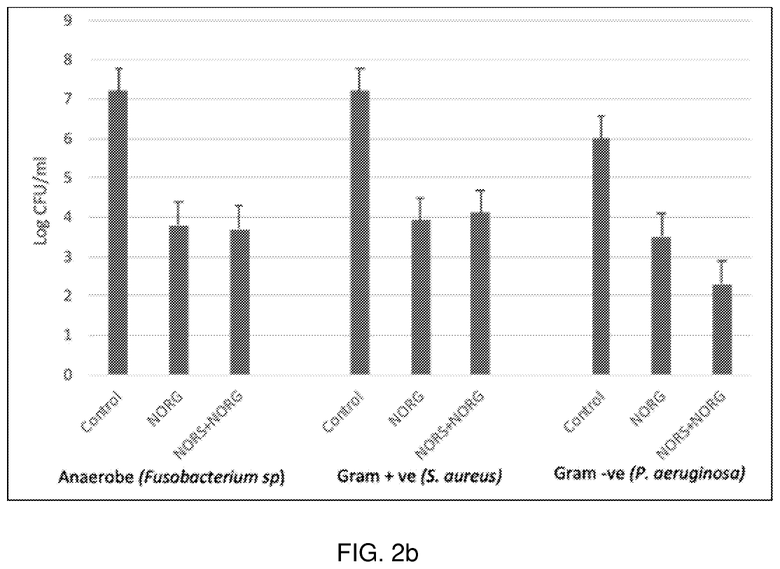

As illustrated in FIGS. 2a and 2b, a porcine study was conducted to assess the antimicrobial activity of nitric oxide releasing solution (NORS) and nitric oxide releasing gel (NORG) compared to non-treated control wounds.

A pathogen-free, female, commercially raised Yorkshire-cross having a weight of from about 55 kg (+/−5 kg) was the subject of the study. Partial thickness wounds were created having a custom-designed 2.0 cm diameter, as illustrated in FIG. 2a. A bacterial mixture innoculum (e.g., Fusobacterium sp., Staphylococcus aureus (ATCC 6538), and Pseudomonas aeruginosa (pig isolate)) was administered in a sterile irrigating bulb filled with 60-80 mL of inoculum and used to flood the gauze until the gauze became saturated with the bacterial mixture. The gauze strip was covered with an occlusive layer (S.C. Johnson and Sons, Brantford, ON) and left on for 1 hour.

The wound intervention involved a treatment 1 or a treatment 2. Treatment 1 used NORG in a volume of about 2 to 5 mL which is equivalent to an exposure of 104 to 210 ppm*min and 52 ppm*min/mL. This NORG treatment was followed by NORS saturated gauze having a volume of 2 to 5 mL which is equivalent to 26 to 65 ppm*min and 13 ppm*min/mL. This NORS treatment was followed by covering with a clear occlusive dressing and incubating for 7 hours.

Treatment 2 involved a NORS rinse of a volume of 1 mL which is equivalent to 13 ppm*min exposure. This was following by NORG in a volume of about 2 to 5 mL which is equivalent to an exposure of 104 to 210 ppm*min and 52 ppm*min/mL. This NORG treatment was followed by NORS saturated gauze having a volume of 2 to 5 mL which is equivalent to 26 to 65 ppm*min and 13 ppm*min/mL. This NORS treatment was followed by covering with a clear occlusive dressing and incubating for 7 hours.

The control involved covering the wound with a piece of sterile, saline-moistened, non-adherent dressing (Curad) and secured with Transpore tape and incubating for 7 hours.

FIG. 2b provides a chart of results depicting the concentration (Log CFU/mL) of different bacteria (Fusobacterium sp., Staphylococcus aureus, and Pseudomonas aeruginosa) within the wound 7.5 hours post treatment with either—NORG alone or wash with NORS and then NORG.

In the case of Fusobacterium sp., the concentration of the bacteria was about 7.2 Log CFU/mL for the control, about 3.8 Log CFU/mL for NORG alone, and about 3.7 Log CFU/mL for wash with NORS and then NORG. These results indicate that NORG alone or NORS and then NORG reduce the concentration of Fusobacterium sp significantly compared to control.

In the case of Staphylococcus aureus, the concentration of the bacteria was about 7.2 Log CFU/mL for the control, about 3.9 Log CFU/mL for NORG alone, and about 4.1 Log CFU/mL for wash with NORS and then NORG. These results indicate that NORG alone or NORS and then NORG reduce the concentration of Staphylococcus aureus significantly compared to control.

In the case of Pseudomonas aeruginosa, the concentration of the bacteria was about 6.0 Log CFU/mL for the control, about 3.5 Log CFU/mL for NORG alone, and about 2.2 Log CFU/mL for wash with NORS and then NORG. These results indicate that NORG alone or NORS and then NORG reduce the concentration of Staphylococcus aureus significantly compared to control. These results also indicate that treatment with NORS and then NORG reduces the concentration of Staphylococcus aureus more so than NORG alone.

### Example 3b: Warts

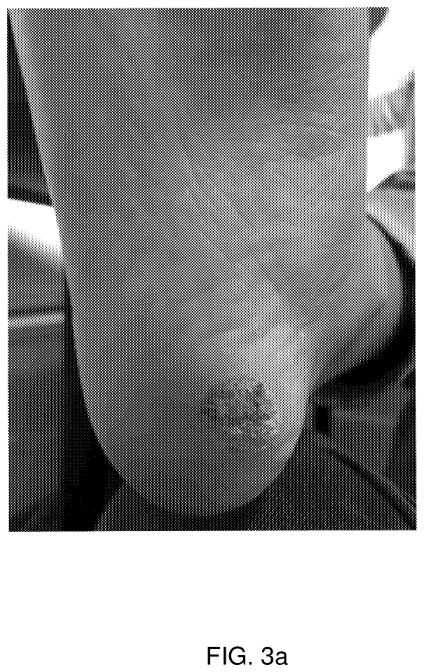

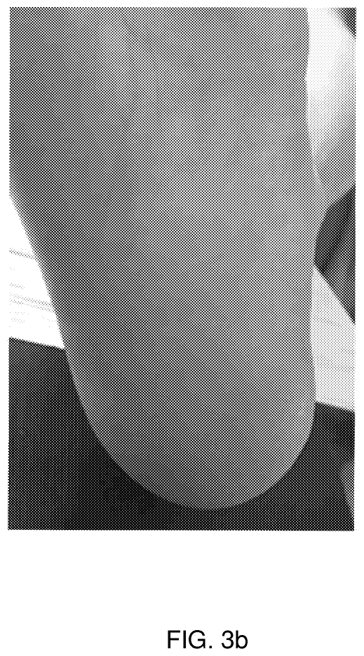

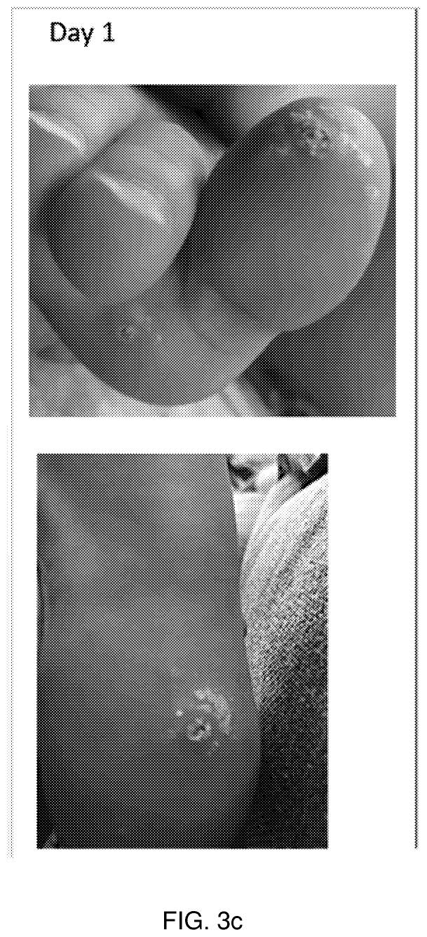

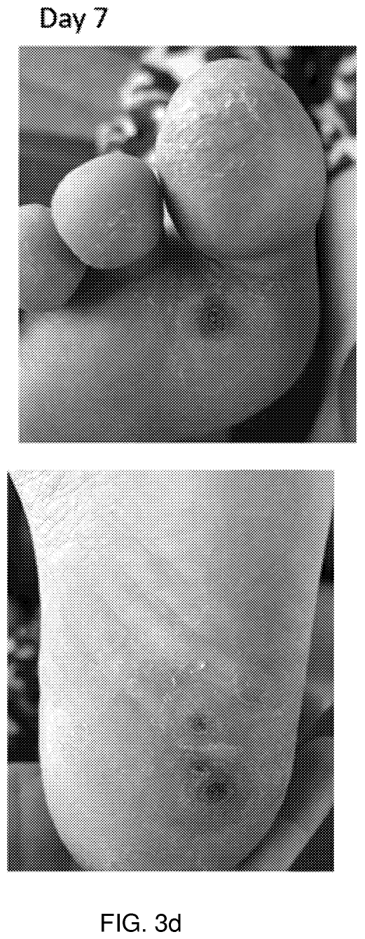

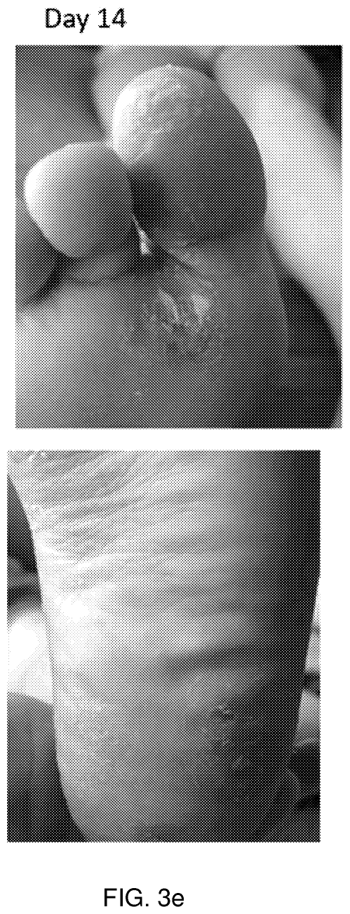

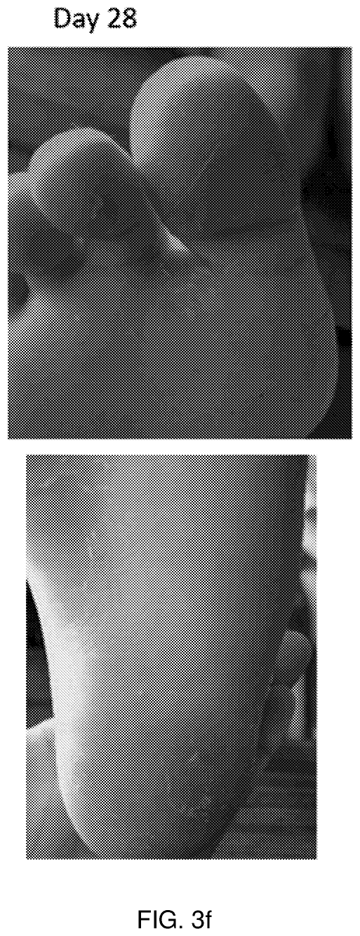

As illustrated in FIGS. 3a to 3f, a study was conducted on a human subject to assess the effects of nitric oxide releasing solution (NORS) and nitric oxide releasing gel (NORG) on warts. A foot of a human subject presented warts in 3 areas: (a) the underside of the big toe, (b) the underside of the back left part of the foot, and (c) a portion on the underside below the big toe. The affected foot was treated for a total of 3 half-hour NORS foot baths (12.85 ppm*min/mL) per week for two weeks. The affected foot was also treated for a duration of one week with once per day application of NORG (52 ppm*min/mL).

As illustrated in FIG. 3a, the wart on the underside of the back left part of the foot was substantial before treatment and was completely removed 28 days after the commencement of treatment, as illustrated in FIG. 3b. FIGS. 3c to 3f illustrated the progression of treatment on day 1 (FIG. 3c), day 7 (FIG. 3d), day 14 (FIG. 3e), and day 28 (FIG. 3f). These results indicate that treatment with NORS and NORG can remove warts on a foot over the course of 28 days.

### Example 3c: Nail Fungus

### Case Study 3c-1

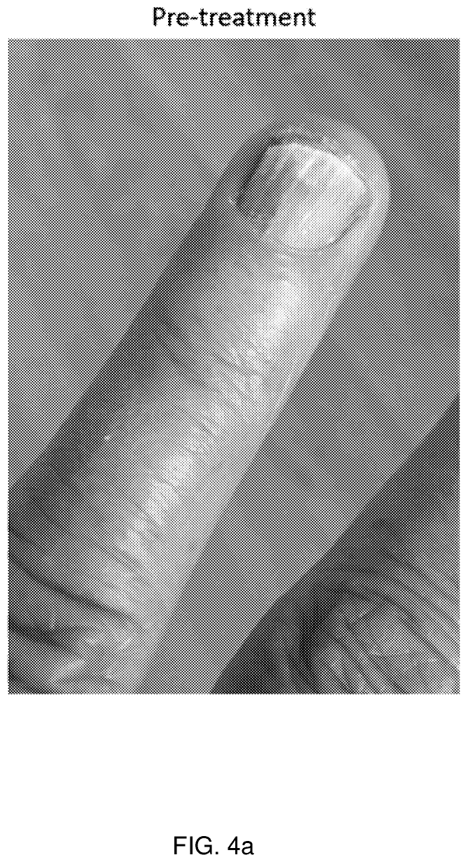

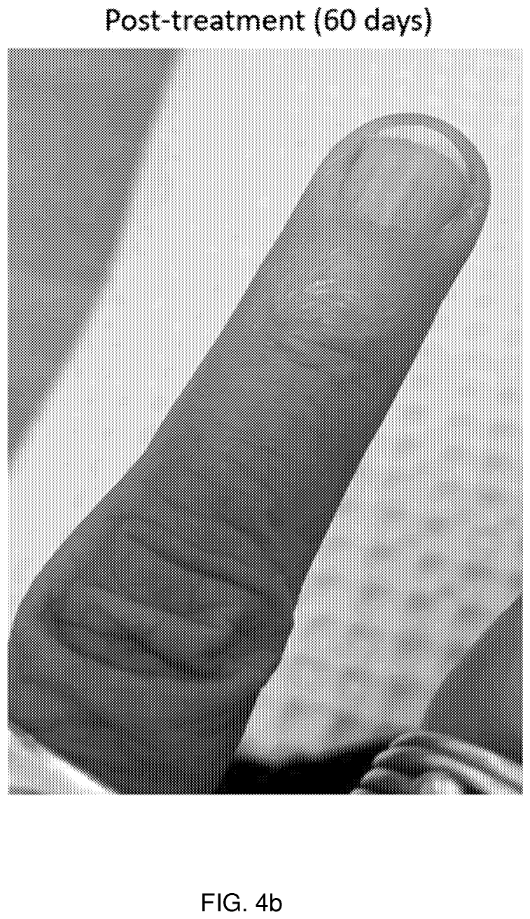

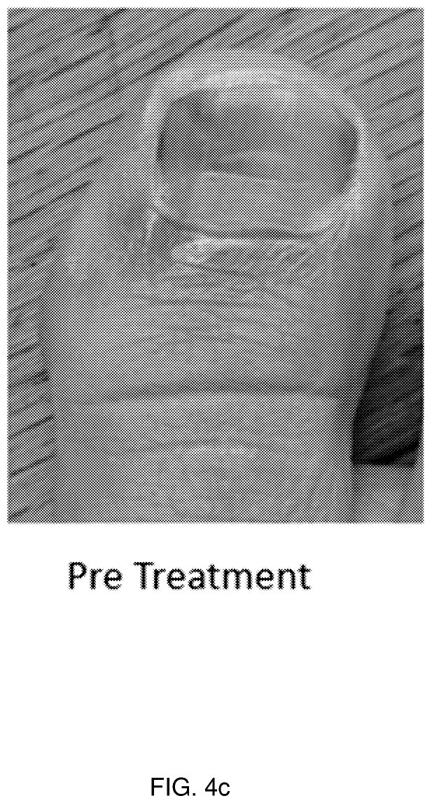

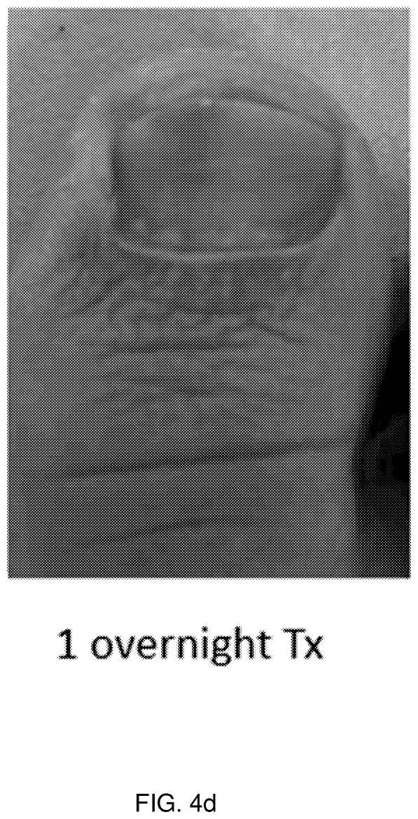

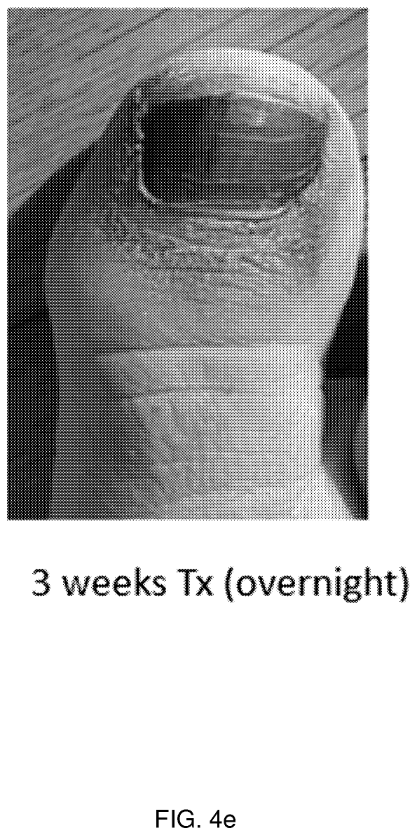

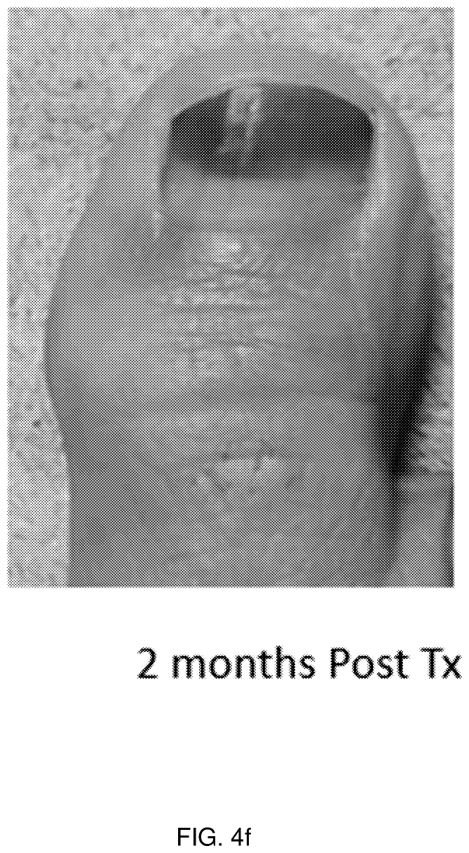

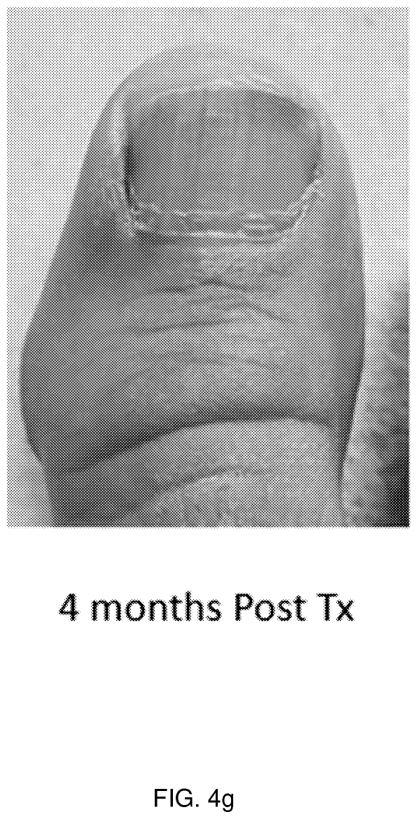

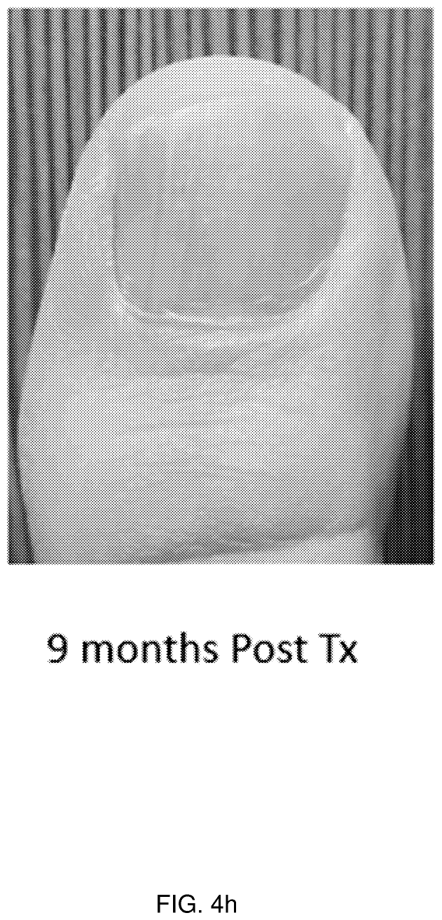

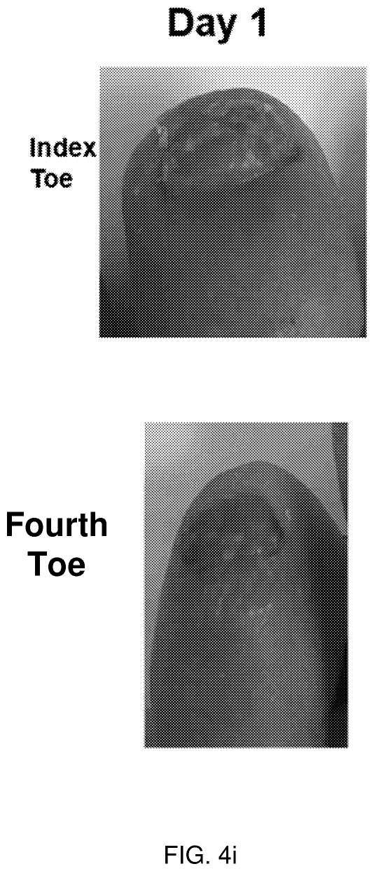

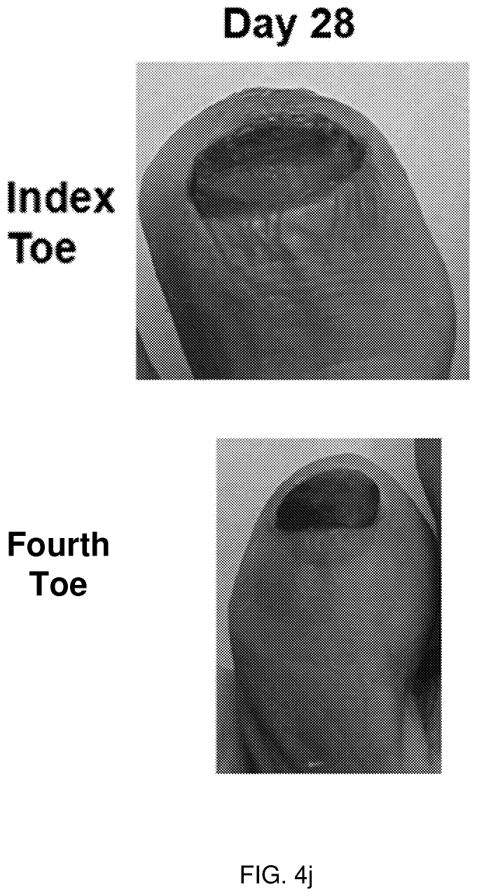

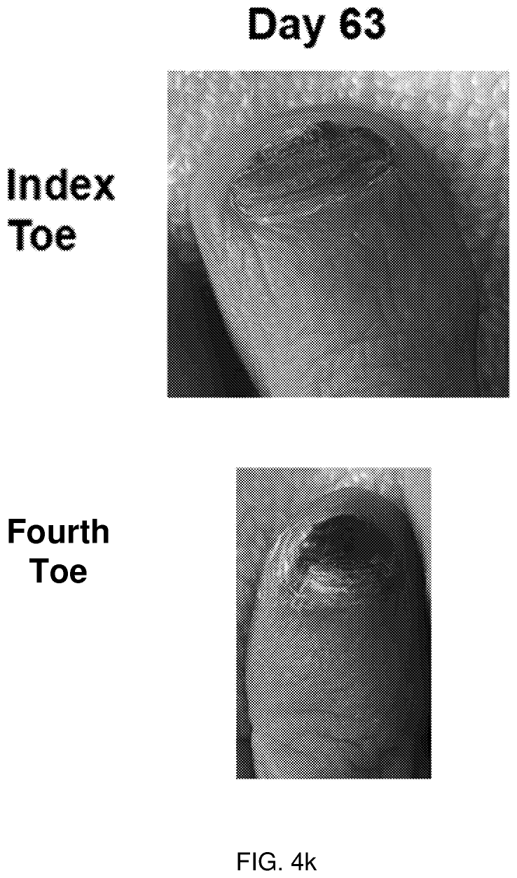

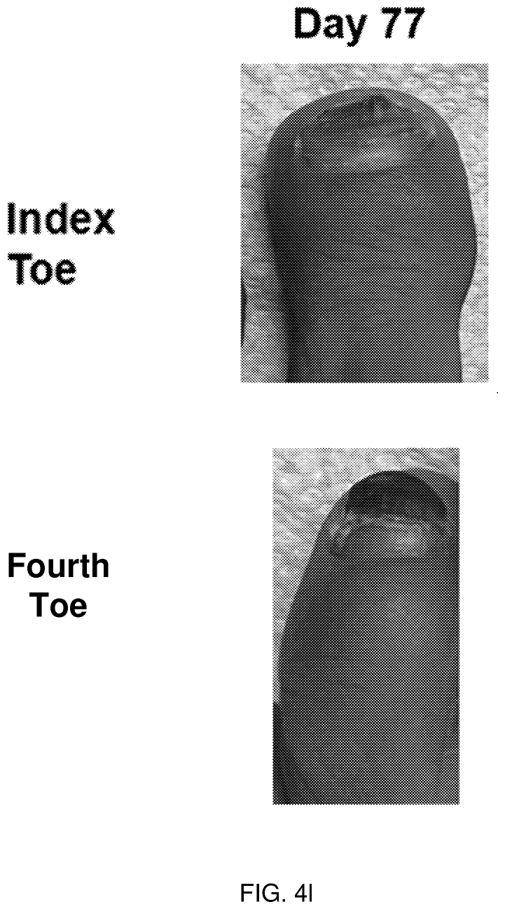

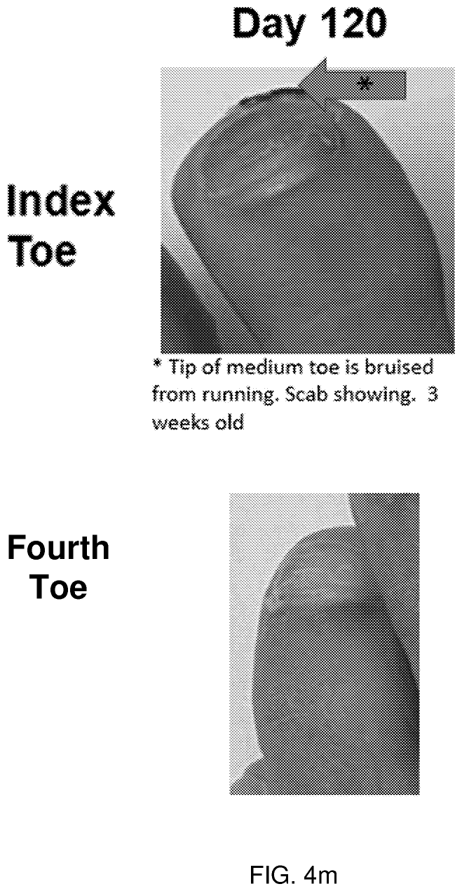

As illustrated in FIGS. 4a and 4b, a study was conducted on a human subject to assess the effects of nitric oxide releasing solution (NORS) and nitric oxide releasing gel (NORG) on nail fungus. A finger of a human subject presented nail fungus. The affected finger was treated for a total of 2 weeks with daily 1 mL of NORG (52 ppm*min/mL).

As illustrated in FIG. 4a, the nail fungus before the commencement of treatment covers approximately ⅓ of the finger nail and was completely removed 60 days after the commencement of treatment, as illustrated in FIG. 4b. These results indicate that treatment with NORS and NORG can remove nail fungus on a finger over the course of 60 days.

### Case Study 3c-2

As illustrated in FIGS. 4c to 4h, a study was conducted on a human subject to assess the effects of nitric oxide releasing solution (NORS) and nitric oxide releasing gel (NORG) on nail fungus on a toe. A toe of a human subject presented nail fungus. The affected toe was treated for a total of 3 weeks with daily 1 mL of NORG (52 ppm*min/mL).

As illustrated in FIG. 4c, the nail fungus before the commencement of treatment covered approximately ½ of the toenail and was completely removed 9 months after the commencement of treatment, as illustrated in FIG. 4h. FIGS. 4c to 4g illustrated the progression of treatment on day 1 (FIG. 4d), day 21 (FIG. 4e), month 2 (FIG. 4f), and month 4 (FIG. 4g). These results indicate that treatment with NORS and NORG can remove nail fungus on a finger over the course of 9 months.

### Case Study 3c-3.

As illustrated in FIGS. 4i and 4m, a study was conducted on a human subject to assess the effects of nitric oxide releasing solution (NORS) and nitric oxide releasing gel (NORG) on nail fungus on an index toe and a fourth toe. An index toe and fourth toe of a human subject presented nail fungus. The affected toes were treated for a total of 4 weeks with daily 1 mL of NORG (52 ppm*min/mL).

As illustrated in FIG. 4i, the nail fungus on day 1 of treatment covered the entire toenail of the index toe and the fourth toe and was completely removed 120 days after the commencement of treatment, as illustrated in FIG. 4m. FIGS. 4j to 4l illustrated the progression of treatment on day 28 (FIG. 4j), day 63 (FIG. 4k), and day 77 (FIG. 4l). These results indicate that treatment with NORS and NORG can remove nail fungus on toes over the course of 120 days.

### EXAMPLE EMBODIMENTS

The following examples pertain to specific technology embodiments and point out specific features, elements, or steps that may be used or otherwise combined in achieving such embodiments.

In one example, there is provided a method of treating a condition responsive to nitric oxide (NO) treatment/therapy in a subject.

In one example, a method of treating a condition responsive to NO treatment/therapy can include administering a volume of a nitric oxide releasing solution (NORS) that provides a therapeutically effective amount of NO to an administration situs of the subject for a treatment duration.

In one example, a method of treating a condition responsive to NO treatment/therapy can include a composition. In one example, the composition can be NORS that can include a nitrite and an acidifying agent. In one example, when measured by releasing NO from the NORS for a period of 30 minutes into a flow of substantially inert carrier gas at a flow rate of 1 liter per minute and a pressure of 1 atmosphere, coupled to a chemiluminescence detector, the NORS can release NO in an amount of from about 0.05 ppm*min to about 500 ppm*min per mL of the NORS.

In one example, a method of treating a condition responsive to NO treatment/therapy can include a composition. In one example, the composition can be NORS that can release NO in an amount from about 0.05 ppm*min to about 10 ppm*min per mL of the NORS. In one example, the volume of the NORS can be from about 0.1 mL to about 10,000 mL. In one example, the total amount of NO released by the volume of NORS administered can be from about 0.1 ppm*min to about 3,000 ppm*min.

In one example, a method of treating a condition responsive to NO treatment/therapy can include a condition that can be a wound, a skin infection, a fungal infection, a respiratory infection, a bacterial infection, or a viral infection.

In one example, a method of treating a condition responsive to NO treatment/therapy can include a condition that can be athlete's foot. In one example, there is provided a method of treating athlete's foot. In one example, the administration situs can be a foot of the subject. In one example, the NORS can release NO in an amount of from about 0.2 ppm*min to about 5 ppm*min per mL of the NORS. In one example, the total amount of NO released by the volume of NORS administered can be from about 1,000 ppm*min to about 5,000 ppm*min. In one example, the volume of NORS administered can be from about 500 mL to about 10,000 mL. In one example, the volume of NORS administered can release a peak NO concentration of from about 5,000 ppm to about 20,000 ppm.

In one example, a method of treating a condition responsive to NO treatment/therapy can include a condition that can be onychomycosis. In one example, there is provided a method of treating a condition that can include onychomycosis. In one example, the administration situs can be a nail of the subject. In one example, the NORS can release NO in an amount of from about 1 ppm*min to about 2 ppm*min per mL of the NORS. In one example, the total amount of NO released by the volume of NORS administered can be from about 1 ppm*min to about 4 ppm*min. In one example, the volume of NORS administered can be from about 0.5 mL to about 2 mL. In one example, the volume of NORS administered can release a peak NO concentration of from about 5 ppm to about 30 ppm.

In one example, a method of treating a condition responsive to NO treatment/therapy can include a condition that can be influenza. In one example, there is provided a method of treating a condition that can be influenza. In one example, the method of treatment can include an administration situs that can be a nasal cavity of the subject. In one example, the NORS can release NO in an amount of from about 0.1 ppm*min to about 10 ppm*min per mL of the NORS. In one example, the total amount of NO released by the volume of NORS administered can be from about 5 ppm*min to about 10 ppm*min. In one example, the volume of NORS administered can be from about 0.5 mL to about 1 mL. In one example, the volume of NORS administered can release a peak NO concentration of from about 1 ppm to about 4 ppm.

In one example, a method of treating a condition responsive to NO treatment/therapy can include a condition that can be a respiratory viral infection. In one example, there is provided a method of treating a condition that can be a respiratory viral infection and the administration situs can be a nasal cavity or lungs of the subject. In one example, the NORS can release NO in an amount of from about 10 ppm*min to about 15 ppm*min per mL of the NORS. In one example, the total amount of NO released by the volume of NORS administered is from about 5 ppm*min to about 10 ppm*min. In one example, the volume of NORS administered can be from about 0.5 mL to about 1 mL. In one example, the volume of NORS administered can release a peak NO concentration of from about 1 ppm to about 4 ppm.

In one example, a method of treating a condition responsive to NO treatment/therapy can include a condition that can be sinusitis. In one example, there is provided a method of treating a condition that can be sinusitis and the administration situs can be a sinus cavity of the subject. In one example, the NORS can release NO in an amount of from about 1 ppm*min to about 15 ppm*min per mL of the NORS. In one example, the total amount of NO released by the volume of NORS administered can be from about 400 ppm*min to about 3,200 ppm*min. In one example, the volume of NORS administered can be from about 150 mL to about 300 mL. In one example, the volume of NORS administered can release a peak NO concentration of from about 100 ppm to about 1,200 ppm.

In one example, a method of treating a condition responsive to NO treatment/therapy can include a condition that can be a throat infection. In one example, there is provided a method of treating a condition that can be a throat infection and the administration situs can be the throat of the subject. In one example, the NORS can release NO in an amount of from about 15 ppm*min to about 30 ppm*min per mL of the NORS. In one example, the total amount of NO released by the volume of NORS administered can be from about 400 ppm*min to about 600 ppm*min. In one example, the volume of NORS administered can be from about 10 mL to about 20 mL. In one example, the volume of NORS administered can release a peak NO concentration of from about 100 ppm to about 200 ppm.

In one example, a method of treating a condition responsive to NO treatment/therapy can include a condition that can be acne. In one example, there is provided a method of treating a condition that can be acne and the administration situs can be a skin area of the subject. In one example, the NORS can release NO in an amount of from about 10 ppm*min to about 30 ppm*min per mL of the NORS. In one example, the total amount of NO released by the volume of NORS administered can be from about 10 ppm*min to about 60 ppm*min. In one example, the total amount of NO released by the volume of NORS administered can be from about 20 ppm*min to about 60 ppm*min. In one example, the volume of NORS administered can be from about 1 mL to about 5 mL. In one example, the volume of NORS administered can release a peak NO concentration of from about 5 ppm to about 20 ppm.

In one example, a method of treating a condition responsive to NO treatment/therapy can include a condition that can be a diabetic foot ulcer. In one example, there is provided a method of treating a condition that can be a diabetic foot ulcer and the administration situs can be a foot of the subject. In one example, the NORS can release NO in an amount of from about 5 ppm*min to about 80 ppm*min per mL of the NORS. In one example, the NORS can release NO in an amount of from about 10 ppm*min to about 60 ppm*min per mL of the NORS. In one example, the total amount of NO released by the volume of NORS administered can be from about 100 ppm*min to about 75,000 ppm*min. In one example, the total amount of NO released by the volume of NORS administered can be from about 200 ppm*min to about 75,000 ppm*min. In one example, the volume of NORS administered can be from about 5 mL to about 10,000 mL. In one example, the volume of NORS administered can release a peak NO concentration of from about 50 ppm to about 20,000 ppm.

In one example, a method of treating a condition responsive to NO treatment/therapy can include a condition that can be warts. In one example, there is provided a method of treating a condition that can be warts and the administration situs can be a skin area of the subject. In one example, the NORS can release NO in an amount of from about 10 ppm*min to about 60 ppm*min per mL of the NORS. In one example, the total amount of NO released by the volume of NORS administered can be from about 45 ppm*min to about 75 ppm*min. In one example, the total amount of NO released by the volume of NORS administered can be from about 20 ppm*min to about 400 ppm*min. In one example, the volume of NORS administered can be from about 0.5 mL to about 10 mL. In one example, the volume of NORS administered can release a peak NO concentration of from about 1 ppm to about 60 ppm.

In one example, a method of treating a condition responsive to NO treatment/therapy can include a composition. In one example, the composition can be NORS that can include at least one NO releasing compound and at least one acidifying agent. In one example, the NO releasing compound can include a nitrite. In one example, the nitrite can be sodium nitrite, potassium nitrite, barium nitrite, calcium nitrite, nitrite orotate, amyl nitrite, magnesium nitrite, silver nitrite, copper nitrite, or a combination thereof. In further examples, the acidifying agent comprises ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof.

In one example, a method of treating a condition responsive to NO treatment/therapy can include a composition. In one example, the composition can be NORS that can provide an extended release of NO.

In one example, a method of treating a condition responsive to NO treatment/therapy can include a composition. In one example, the composition can comprise NORS that can have an amount of NO released by the NORS can be measured by: introducing the flow of the carrier gas into a sample holding chamber via a gas inlet; introducing the NORS into the sample holding chamber via a sample inlet to contact the continuous flow of carrier gas; directing the carrier gas and any released NO out of the sample holding chamber via an outlet to an NO detector; and quantifying the amount of released NO using the NO detector. In one example, the sample holding chamber can include an interior surface that is inert with respect to NO.

In one example, a method of treating a condition responsive to NO treatment/therapy can include NORS that can be administered as an intranasal spray, an open vessel bath for soaking, a liquid sinus wash, a liquid gargling solution, a respiratory inhaler, an aerosol, a gel, a foam, an emulsion, a liquid solution, a cream, or a combination thereof.

In one example, a method of treating a condition responsive to NO treatment/therapy can include a composition. In one example, the composition can be NORS that can include a pharmaceutically acceptable carrier. In one example, the pharmaceutically acceptable carrier can include water, saline, hydroxyethyl cellulose, xanthan gum, or a combination thereof.

In one example, a composition for treating a condition responsive to nitric oxide (NO) treatment can include a nitric oxide releasing solution (NORS). In one example, the NORS can include at least one NO releasing compound and at least one acidifying agent. In one example, when measured by releasing NO from the NORS for a period of 30 minutes into a flow of substantially inert carrier gas at a flow rate of 1 liter per minute and a pressure of 1 atmosphere, coupled to a chemiluminescence detector, the NORS can release NO in an amount of from about 0.05 ppm*min to about 500 ppm*min per mL of the NORS.

In one example, a method of treating a condition responsive to NO treatment/therapy can include a composition. In one example, the composition can be formulated to be administered to a foot of a subject to treat athlete's foot, and the NORS can release NO in an amount of from about 10 ppm*min to about 15 ppm*min per mL of the NORS.

In one example, a method of treating a condition responsive to NO treatment/therapy can include a composition. In one example, the composition can be formulated to be administered to a nail of a subject to treat onychomycosis, and the NORS can release NO in an amount of from about 45 ppm*min to about 60 ppm*min per mL of the NORS.

In one example, a method of treating a condition responsive to NO treatment/therapy can include a composition. In one example, the composition can be formulated to be administered to a nasal cavity of a subject to treat influenza, and the NORS can release NO in an amount of from about 10 ppm*min to about 15 ppm*min per mL of the NORS.

In one example, a method of treating a condition responsive to NO treatment/therapy can include a composition. In one example, the composition can be formulated to be administered to a nasal cavity or lungs of a subject to treat a respiratory viral infection, and the NORS can release NO in an amount of from about 10 ppm*min to about 15 ppm*min per mL of the NORS.

In one example, a method of treating a condition responsive to NO treatment/therapy can include a composition. In one example, the composition can be formulated to be administered to a sinus cavity of a subject to treat sinusitis, and the NORS can release NO in an amount of from about 1 ppm*min to about 15 ppm*min per mL of the NORS.

In one example, a method of treating a condition responsive to NO treatment/therapy can include a composition. In one example, the composition can be formulated to be administered to a throat of a subject to treat a throat infection, and the NORS can release NO in an amount of from about 15 ppm*min to about 30 ppm*min per mL of the NORS.

In one example, a method of treating a condition responsive to NO treatment/therapy can include a composition. In one example, the composition can be formulated to be administered to a skin area of a subject to treat acne, and the NORS can release NO in an amount of from about 10 ppm*min to about 30 ppm*min per mL of the NORS.

In one example, a method of treating a condition responsive to NO treatment/therapy can include a composition. In one example, the composition can be formulated to be administered to a foot of a subject to treat a diabetic foot ulcer, and the NORS can release NO in an amount of from about 10 ppm*min to about 60 ppm*min per mL of the NORS.

In one example, a method of treating a condition responsive to NO treatment/therapy can include a composition. In one example, the composition can be formulated to be administered to a skin area of a subject to treat warts, and the NORS can release NO in an amount of from about 10 ppm*min to about 60 ppm*min per mL of the NORS. In one example, the composition can be formulated to be administered to a skin area of a subject to treat warts, and the NORS can release NO in an amount of from about 5 ppm*min to about 80 ppm*min per mL of the NORS.

In one example, a method of treating a condition responsive to NO treatment/therapy can include a composition. In one example, the composition can be an NO releasing compound that can include a nitrite. In one example, the nitrite can be sodium nitrite, potassium nitrite, barium nitrite, calcium nitrite, nitrite orotate, amyl nitrite, magnesium nitrite, silver nitrite, copper nitrite, or a combination thereof.

In one example, a method of treating a condition responsive to NO treatment/therapy can include a composition. In one example, the composition can include an acidifying agent. In one example, the acidifying agent can include ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof.

In one example, a method of treating a condition responsive to NO treatment/therapy can include a composition. In one example, the composition can be NORS that can provide an extended release of NO.

In one example, a method of treating a condition responsive to NO treatment/therapy can include a composition. In some examples, the composition can include NORS, wherein an amount of NO released by the NORS can be measured by: introducing the flow of the carrier gas into a sample holding chamber via a gas inlet; introducing the NORS into the sample holding chamber via a sample inlet to contact the continuous flow of carrier gas; directing the carrier gas and any released NO out of the sample holding chamber via an outlet to an NO detector; and quantifying the amount of released NO using the NO detector. In one example, the sample holding chamber can include an interior surface that is inert with respect to NO.

In one example, a method of treating a condition responsive to NO treatment/therapy can include a composition. In one example, the composition can be formulated to be administered as an intranasal spray, an open vessel bath for soaking, a liquid sinus wash, a liquid gargling solution, a respiratory inhaler, an aerosol, a gel, a foam, an emulsion, a liquid solution, a cream, or a combination thereof.

In one example, a method of treating a condition responsive to NO treatment/therapy can include a composition. In one example, the composition can include a pharmaceutically acceptable carrier. In one example, the pharmaceutically acceptable carrier can comprise water, saline, hydroxyethyl cellulose, xanthan gum, or a combination thereof.

In one example, a method of treating a condition responsive to NO treatment/therapy can include a condition that can be athlete's foot. In one example, a method of treating athlete's foot in a subject can include: administering a nitric oxide releasing solution (NORS) that provides a therapeutically effective dose of nitric oxide (NO) gas to a foot of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes, the NORS releases NO in an amount of from about 50,000 ppm*min to about 75,000 ppm*min. In one example, the volume of NORS administered can be from about 1,000 mL to about 10,000 mL. In one example, the NORS can release NO in an amount of from about 5 ppm*min to about 15 ppm*min per mL of the NORS. In one example, the NORS can release NO in an amount of from about 10 ppm*min to about 15 ppm*min per mL of the NORS. In one example, the NORS can release NO in an amount of from about 25 ppm*min to about 500 ppm*min per mL of the NORS. In one example, the volume of NORS administered can release a peak NO concentration of from about 5,000 ppm to about 20,000 ppm. In one example, the volume of NORS administered can release a peak NO concentration of from about 15,000 ppm to about 20,000 ppm. In one example, the NORS can be administered as a liquid solution in a foot bath. In one example, the method includes administering the NORS according to a dosage regimen of soaking the foot in the foot bath for 30 minutes once every 4 days. In one example, the NORS can comprise at least one NO releasing compound and at least one acidifying agent. In one example, the NO releasing compound can comprise a nitrite. In one example, the nitrite can be sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be onychomycosis in a nail of a subject. In one example, the method can include: administering a nitric oxide releasing solution (NORS) that can provide a therapeutically effective dose of nitric oxide (NO) gas to the nail of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 45 ppm*min to about 60 ppm*min. In one example, the volume of NORS administered can be from about 0.5 mL to about 2 mL. In one example, the NORS can release the NO in an amount of from about 25 ppm*min to about 75 ppm*min per mL of the NORS. In one example, the NORS can release the NO in an amount of from about 45 ppm*min to about 60 ppm*min per mL of the NORS. In one example, the volume of NORS administered can release a peak NO concentration of from about 5 ppm to about 15 ppm. In one example, the NORS can be administered as a liquid solution, a gel, or a cream. In one example, the method can include administering the NORS according to a dosage regimen of applying NORS to the nail once every 21 days. In one example, the NORS can comprise at least one NO releasing compound and at least one acidifying agent. In one example, the NO releasing compound can comprise a nitrite. In one example, the nitrite can comprise sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be influenza. In one example, a method of treating influenza in a subject can include: administering a nitric oxide releasing solution (NORS) that provides a therapeutically effective dose of nitric oxide (NO) gas to a nasal cavity of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 5 ppm*min to about 10 ppm*min. In one example, the volume of NORS administered can be from about 0.5 mL to about 1 mL. In one example, the NORS can release NO in an amount of from about 2 ppm*min to about 15 ppm*min per mL of the NORS. In one example, the NORS can release NO in an amount of from about 10 ppm*min to about 15 ppm*min per mL of the NORS. In one example, the volume of NORS administered can release a peak NO concentration of from about 1 ppm to about 4 ppm. In one example, the NORS can be administered as a nasal spray. In one example, the method can include administering the NORS according to a dosage regimen of administering the nasal spray as needed. In one example, the NORS can comprise at least one NO releasing compound and at least one acidifying agent. In one example, the NO releasing compound can comprise a nitrite. In one example, the nitrite can be sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be a respiratory viral infection. In one example, a method of treating a respiratory viral infection in a subject can include: administering a nitric oxide releasing solution (NORS) that provides a therapeutically effective dose of nitric oxide (NO) gas to a nasal cavity or lungs of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 5 ppm*min to about 10 ppm*min. In one example, the volume of NORS administered can be from about 0.5 mL to about 1 mL. In one example, the volume of NORS administered can be from about 0.5 mL to about 5 mL. In one example, the NORS can release NO in an amount of from about 5 ppm*min to about 15 ppm*min per mL of the NORS. In one example, the NORS can release NO in an amount of from about 10 ppm*min to about 15 ppm*min per mL of the NORS. In one example, the NORS can release NO in an amount of from about 10 ppm*min to about 30 ppm*min per mL of the NORS. In one example, the volume of NORS administered can release a peak NO concentration of from about 1 ppm to about 4 ppm. In one example, the NORS can be administered as a nasal spray. In one example, the method can include administering the NORS according to a dosage regimen of administering the nasal spray as needed. In one example, the respiratory viral infection can be a common cold, respiratory syncytial virus, or corona virus. In one example, the NORS can comprise at least one NO releasing compound and at least one acidifying agent. In one example, the NO releasing compound can comprise a nitrite. In one example, the nitrite can be sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be sinusitis. In one example, a method of treating sinusitis in a subject can include: administering a nitric oxide releasing solution (NORS) that provides a therapeutically effective dose of nitric oxide (NO) gas to a sinus cavity of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 400 ppm*min to about 3,200 ppm*min. In one example, the volume of NORS administered can be from about 150 mL to about 300 mL. In one example, the NORS can release NO in an amount of from about 1 ppm*min to about 15 ppm*min per mL of the NORS. In one example, the NORS can release NO in an amount of from about 1 ppm*min to about 30 ppm*min per mL of the NORS. In one example, the volume of NORS administered can release a peak NO concentration of from about 50 ppm to about 500 ppm. In one example, the volume of NORS administered can release a peak NO concentration of from about 100 ppm to about 1,200 ppm. In one example, the volume of NORS administered can release a peak NO concentration of from about 100 ppm to about 5,000 ppm. In one example, the NORS can be administered as rinse, lavage, or spray. In one example, the method can include administering the NORS according to a dosage regimen of administering the NORS once about every 42 days. In one example, the method can include administering the NORS according to a dosage regimen of administering the NORS daily for a period of about 10 to about 50 days. In one example, the NORS can comprise at least one NO releasing compound and at least one acidifying agent. In one example, the NO releasing compound can comprise a nitrite. In one example, the nitrite can be sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be a throat infection. In one example, a method of treating a throat infection in a subject can include: administering a nitric oxide releasing solution (NORS) that provides a therapeutically effective dose of nitric oxide (NO) gas to the throat of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 400 ppm*min to about 600 ppm*min. In one example, the volume of NORS administered can be from about 10 mL to about 20 mL. In one example, the volume of NORS administered can be from about 5 mL to about 40 mL. In one example, the NORS can release the NO in an amount of from about 15 ppm*min to about 30 ppm*min per mL of the NORS. In one example, the NORS can release the NO in an amount of from about 10 ppm*min to about 40 ppm*min per mL of the NORS. In one example, the volume of NORS administered can release a peak NO concentration of from about 100 ppm to about 200 ppm. In one example, the volume of NORS administered can release a peak NO concentration of from about 200 ppm to about 800 ppm. In one example, the NORS can be administered as a gargling liquid solution. In one example, the method can include administering the NORS according to a dosage regimen of gargling for 1.5 minutes every 3 days. In one example, the NORS can comprise at least one NO releasing compound and at least one acidifying agent. In one example, the NO releasing compound can comprise a nitrite. In one example, the nitrite can comprise sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be acne. In one example, a method of treating acne in a subject can include: administering a nitric oxide releasing solution (NORS) that provides a therapeutically effective dose of nitric oxide (NO) gas to a skin area of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 20 ppm*min to about 60 ppm*min. In one example, the volume of NORS administered can be from about 1 mL to about 5 mL. In one example, the NORS can release NO in an amount of from about 10 ppm*min to about 30 ppm*min per mL of the NORS. In one example, the volume of NORS administered can release a peak NO concentration of from about 5 ppm to about 20 ppm. In one example, the NORS can be administered as cream. In one example, the NORS can comprise at least one NO releasing compound and at least one acidifying agent. In one example, the NO releasing compound can comprise a nitrite. In one example, the nitrite can comprise sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be a diabetic foot ulcer. In one example, a method of treating a diabetic foot ulcer in a subject can include: administering a nitric oxide releasing solution (NORS) that provides a therapeutically effective dose of nitric oxide (NO) gas to a foot of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 200 ppm*min to about 75,000 ppm*min. In one example, the volume of NORS administered can be from about 5 mL to about 10,000 mL. In one example, the NORS can release NO in an amount of from about 10 ppm*min to about 60 ppm*min per mL of the NORS. In one example, the volume of NORS administered can release a peak NO concentration of from about 50 ppm to about 20,000 ppm. In one example, the NORS can be administered as a liquid solution in a foot bath. In one example, the method can include administering the NORS according to a dosage regimen of soaking the foot in the foot bath for 30 minutes once every 12 days. In one example, the NORS can comprise at least one NO releasing compound and at least one acidifying agent. In one example, the NO releasing compound can comprise a nitrite. In one example, the nitrite can comprise sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be warts. In one example, a method of treating warts in a subject can include: administering a nitric oxide releasing solution (NORS) that provides a therapeutically effective dose of nitric oxide (NO) gas to a skin area of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 45 ppm*min to about 75 ppm*min. In one example, the volume of NORS administered can be from about 0.5 mL to about 10 mL. In one example, the NORS can release NO in an amount of from about 10 ppm*min to about 60 ppm*min per mL of the NORS. In one example, the volume of NORS administered can release a peak NO concentration of from about 1 ppm to about 60 ppm. In one example, the NORS can be administered as a liquid solution in a bath. In one example, the method can include administering the NORS according to a dosage regimen of soaking the skin in the bath for 30 minutes once every 6 days. In one example, the method can include administering the NORS according to a dosage regimen of soaking the skin in the bath for 30 minutes daily for a period of about 1 day to about 10 days. In one example, the NORS can comprise at least one NO releasing compound and at least one acidifying agent. In one example, the NO releasing compound can comprise a nitrite. In one example, the nitrite can be sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include a condition that can be skin at risk of infection. In one example, a method of disinfecting skin of a subject can include: administering a nitric oxide releasing solution (NORS) that can provide a therapeutically effective dose of nitric oxide (NO) gas to a skin area of the subject for a treatment duration, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 450 ppm*min to about 750 ppm*min. In one example, the volume of NORS administered can be from about 50 mL to about 200 mL. In one example, the NORS can release the NO in an amount of from about 5 ppm*min to about 25 ppm*min per mL of the NORS. In one example, the volume of NORS administered can release a peak NO concentration of from about 1 ppm to about 20 ppm. In one example, the NORS can comprise at least one NO releasing compound and at least one acidifying agent. In one example, the NO releasing compound can comprise a nitrite. In one example, the nitrite can be sodium nitrite, potassium nitrite, barium nitrite, calcium nitrite, nitrite orotate, amyl nitrite, magnesium nitrite, silver nitrite, copper nitrite, or a combination thereof. In one example, the acidifying agent can comprise ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof. In one example, the NORS can provide an extended release of NO.

In one example, a method of treating a condition responsive to NO treatment/therapy can include the use of a composition as provided herein. In one example, the use of a composition as provided herein for the manufacture of a medicament for treating a condition responsive to nitric oxide (NO) treatment can comprise: administering a therapeutically effective amount of the composition to the subject. In one example, a composition for use in treating a condition responsive to nitric oxide (NO) treatment can comprise: administering a therapeutically effective amount of the composition to a subject.

In one example there is provided a composition, such as a nitric oxide releasing composition.

In one example of a composition, the composition comprises a combination of a nitric oxide releasing compound, an acidifying agent, and a carrier in amounts that release from about 1 ppm*min/mL to about 500 ppm*min/mL nitric oxide (NO) based on 1 mL of the composition when measured by releasing NO from the composition for a period of 30 minutes into a flow of substantially inert carrier gas at a flow rate of 1 liter per minute and a pressure of 1 atmosphere, coupled to a chemiluminescence detector.

In one example of a composition, the pharmaceutically acceptable carrier comprises water, saline, hydroxyethyl cellulose, xanthan gum, or a combination thereof.

In one example of a composition, the composition is formulated to be administered as an intranasal spray, an open vessel bath for soaking, a liquid sinus wash, a liquid gargling solution, a respiratory inhaler, an aerosol, a gel, a foam, an emulsion, a liquid solution, a cream, or a combination thereof.

In one example of a composition, the amount of NO released is from about 1 ppm*min/mL to about 500 ppm*min/mL.

While the forgoing examples are illustrative of the principles of the present technology in one or more particular applications, it will be apparent to those of ordinary skill in the art that numerous modifications in form, usage and details of implementation can be made without the exercise of inventive faculty, and without departing from the principles and concepts of the technology.

What is claimed is:

1. A composition for treating a condition responsive to nitric oxide (NO) treatment comprising a nitric oxide releasing solution (NORS), wherein the NORS includes a nitric oxide releasing compound and an acidifying agent, wherein when measured by releasing NO from the NORS into a flow of substantially inert carrier gas at a flow rate of 1 liter per minute and a pressure of 1 atmosphere to be quantified by a chemiluminescence detector for a period of 30 minutes said NORS releases NO in an amount of from about 1 ppm*min/mL to about 500 ppm*min/mL based on 1 mL of the NORS.

2. The composition of claim 1, wherein the nitric oxide releasing compound comprises sodium nitrite, potassium nitrite, barium nitrite, calcium nitrite, nitrite orotate, amyl nitrite, magnesium nitrite, silver nitrite, copper nitrite, or a combination thereof.

3. The composition of claim 1, wherein the acidifying agent comprises ascorbic acid, ascorbyl palmitate, salicylic acid, malic acid, lactic acid, citric acid, formic acid, benzoic acid, tartaric acid, hydrochloric acid, sulfuric acid, phosphoric acid, or a combination thereof.

4. The composition of claim 1, wherein the NORS provides an extended release of NO.

5. The composition of claim 1, wherein the composition is formulated to be administered as an intranasal spray, an open vessel bath for soaking, a liquid sinus wash, a liquid gargling solution, a respiratory inhaler, an aerosol, a gel, a foam, an emulsion, a liquid solution, a cream, or a combination thereof.

6. The composition of claim 1, wherein the composition further comprises a pharmaceutically acceptable carrier.

7. The composition of claim 6, wherein the pharmaceutically acceptable carrier comprises water, saline, hydroxyethyl cellulose, xanthan gum, or a combination thereof.
